(19) United States
(12) Patent Application Publication
(10) Pub. No.: US 2014/0094407 A1
(43) Pub. Date: Apr. 3, 2014

(54) DEVICES AND METHODS FOR TREATING AND/OR PREVENTING DISEASES

(71) Applicant: Combinent Biomedical Systems, Inc., Lexington, MA (US)

(72) Inventors: Eyal S. Ron, Lexington, MA (US); William F. Crowley, JR., Cambridge, MA (US); Robert S. Langer, Newton, MA (US); Surajit K. Biswas, Bedford, MA (US); Quentin Baca, Lexington, MA (US); Armen Tashjian, Lexington, MA (US)

(73) Assignee: Combinent Biomedical Systems, Inc., Lexington, MA (US)

(21) Appl. No.: 14/096,493

(22) Filed: Dec. 4, 2013

Related U.S. Application Data
(63) Continuation of application No. 13/063,887, filed on May 26, 2011, now abandoned.
(60) Provisional application No. 61/161,502, filed on Mar. 19, 2009.
(60) Provisional application No. 61/155,696, filed on Feb. 26, 2009.
(60) Provisional application No. 61/155,694, filed on Feb. 26, 2009.
(60) Provisional application No. 61/152,304, filed on Feb. 13, 2009.
(60) Provisional application No. 61/112,372, filed on Nov. 7, 2008.
(60) Provisional application No. 61/112,369, filed on Nov. 7, 2008.
(60) Provisional application No. 61/112,366, filed on Nov. 7, 2008.
(60) Provisional application No. 61/112,377, filed on Nov. 7, 2008.

(51) Int. Cl. A61K 47/32; A61K 38/09

(52) U.S. Cl. CPC A61K 47/32; A61K 38/09
USPC 514/5.8; 514/1.1; 514/10.4; 514/11.7; 514/9.7

(57) ABSTRACT

The invention relates to a therapeutic device for the delivery of therapeutic agents, e.g. a peptide such as leuprolide, via the vagina to a female mammal. In some embodiments, the invention also relates to methods for the treatment of obesity and eating disorders, diabetes, multiple sclerosis (MS), endometriosis, uterine fibroids, polycystic ovarian disease, various cancers such as breast cancer, acne, hirsutism, microbial or fungal or viral infections such as bacterial vaginosis or AIDS/HIV, and chronic diseases using a disclosed vaginal device.

### CROSS-REFERENCE TO RELATED APPLICATIONS

This application claims the benefit and priority to U.S. Provisional Application No. 61/152,304, filed Feb. 13, 2009, U.S. Provisional Application No. 61/161,502, filed Mar. 19, 2009, U.S. Provisional Application No. 61/112,366, filed Nov. 7, 2008; U.S. Provisional Application No. 61/155,694, filed Feb. 26, 2009; U.S. Provisional Application No. 61/112,369, filed Nov. 7, 2008; U.S. Provisional Application No. 61/112,372, filed Nov. 7, 2008; U.S. Provisional Application No. 61/112,377, filed Nov. 7, 2008; and U.S. Provisional Application No. 61/155,696, filed Feb. 26, 2009; all of which are hereby incorporated by reference in their entirety.

### BACKGROUND

Vaginal drug delivery has several advantages: (1) it may be non-invasive; (2) the vagina consists of highly perfused tissue with a well-developed blood supply; (3) it may avoid first-pass metabolism in the liver. Further, administration of therapeutic agents via vaginal drug delivery may be beneficial for the treatment of diseases that require painful injections or require therapies that are difficult to comply with.

Constant and reliable delivery of drugs or combinations of drugs over long periods of time, using a vaginal route of administration, may be useful in a wide variety of applications. For example, peptides are attractive therapeutic candidates because of their potential high specificity and low toxicity. However, delivery of such peptides traditionally requires painful, inconvenient and uncomfortable injections.

For example, leuprolide and other GnRH agonists are typically used in female patients for the treatment of breast cancer or estrogen dependent conditions such as endometriosis or uterine fibroids, as well as to control ovarian stimulation during in vitro fertilization. However, leuprolide is administered typically as an intramuscular depot injection (e.g., Lupron Depot®), or a subcutaneous injection, in a formulation that typically includes polylactic acid. Injections can be painful and patient compliance using such a delivery system may be problematic.

Some medications require simultaneous use of contraceptives. For example, isotretinoin is a medication used for the treatment of severe acne, that can cause birth defects if women become pregnant while taking it or take it while pregnant. For this reason, the United States Food and Drug Administration (FDA) has mandated that female patients to use two forms of contraception while on isotretinoin. Antiandrogens are often used for hirsutism, which is defined as abnormal male-pattern hair growth in females, to block the stimulatory effect of testosterone on hair follicles. However, antiandrogen therapies can potentially cause birth defects.

A patient manageable drug delivery device that can release a peptide or other therapeutic, alone or with one, two or three different therapeutic agents, e.g., continuously over time, and without the need for frequent injections or a administration of a depot formulation, would be beneficial, especially for treatment of diseases that require injection of e.g. a peptide therapeutic that may not be amenable to oral therapy.

### SUMMARY

The disclosure is generally directed to a therapeutic device for delivery of one or more active agents such as a therapeutic peptide via the vagina to treat a disorder affecting a female patient, wherein the device comprises at least one segment that includes a peptide permeable thermoplastic polymer, e.g., ethylene-vinyl acetate copolymer, and the active agent (e.g. therapeutic peptide or pharmaceutically acceptable salts thereof), and optionally a pharmaceutically acceptable excipient, e.g., citric acid.

For example, a disclosed therapeutic or drug delivery device that includes a therapeutic peptide may, upon insertion of the device in the vagina of a patient, result in systemic absorption of the therapeutic peptide. Contemplated therapeutic devices can include an unitary segment having a substantially uniform composition that includes the therapeutic peptide and the thermoplastic polymer.

For example, contemplated therapeutic peptides include peptides that are about 4 to about 40 amino acids in length. One exemplary therapeutic peptide is leuprolide or a pharmaceutically acceptable salt thereof. Other contemplated therapeutic peptides can be chosen from exanatide, liraglutide, oxyntomodulin, ghrelin, peptide YY, pramlintide, and pancreatic polypeptide and combinations thereof. In another embodiment, a therapeutic peptide may be chosen from amylin and leptin, and combinations thereof.

Disclosed devices, in some embodiments, after insertion of the device in a female patient may result in a serum level of a disclosed therapeutic peptide or agent after or about 16 hours, about 1 day or more after insertion of said device that is a pharmaceutically effective amount for the treatment of a disorder. For example, peak serum concentration of the therapeutic peptide may be obtained at about 12 to about 22 hours, e.g., at about 16 hours, after insertion of the device. In a certain embodiment, a peak serum concentration of leuprolide in a patient is less, and/or occurs more slowly after insertion of a disclosed device than that of patient administered a leuprolide depot composition by injection.

In a particular embodiment, a disclosed therapeutic device, may, upon insertion of the device, provide a serum level of leuprolide in a patient of about 0.05 ng/mL to about 1.0 ng/mL, for example, about 0.6 ng/mL after about 1 day.

In some embodiments, disclosed therapeutic devices may include a composition that includes about 10 to about 100 mg of a therapeutic peptide, e.g., about 10 to about 60 mg, e.g., about 36 mg, about 54 mg, or about 18 mg of leuprolide acetate. Such a therapeutic device may release about 10 μg/day of leuprolide upon insertion into the vagina of a patient. Such contemplated devices may further comprise progesterin, e.g. may include a second unitary segment comprising progesterin.

Also provided herein is a therapeutic device for the treatment of a female disorder, comprising a unitary segment having a substantially uniform composition, wherein the composition comprises drug permeable ethylene-vinyl-acetate co-polymer and leuprolide or pharmaceutically acceptable salts thereof and optionally a pharmaceutically acceptable excipient, wherein upon insertion of said device in the vagina of a patient results in systemic absorption of leuprolide. In some embodiments, such a device may incur, about 16 hours after insertion of the device in the patient, peak levels of FSH (follicle stimulating hormone) and LH (lutenizing hormone), or wherein peak levels of FSH and LH occur about 10 hours later in a patient as compared to the occurrence of peak levels of FSH and LH after injection of a depot composition comprising leuprolide or pharmaceutically acceptable salts (such as a depot composition that includes about 22.5 mg of leuprolide acetate and polylactic acid) thereof in a patient

The therapeutic devices contemplated herein may include ethylene-vinyl-acetate co-polymer that includes about 4% to about 50% weight percent vinyl acetate, e.g., about 15 to about 40% weight percent vinyl acetate, e.g., about 15 to about 30% vinyl acetate, and/or wherein an ethylene vinyl acetate copolymer has a melt index of 57 g/10 minutes at 190° C./2.16 kg.

Also provided herein is a therapeutic vaginal ring consisting essentially of an unitary segment having a substantially uniform composition, wherein the composition comprises drug permeable ethylene-vinyl-acetate co-polymer and an pharmaceutically effective amount of leuprolide or a pharmaceutically acceptable salt thereof, and optionally, a pharmaceutically acceptable excipient. In another embodiment, a therapeutic vaginal ring formed from one unitary segment is provided that consists essentially of ethylene vinyl acetate co-polymer, an pharmaceutically effective amount of leuprolide or a pharmaceutically acceptable salts thereof, and optionally one or more pharmaceutically acceptable excipients.

In an embodiment, a therapeutic device for delivery of a combination therapy via the vagina, is disclosed, that comprises: a) at least one first segment comprising a drug-permeable thermoplastic polymer and an active agent chosen from an antiandrogen or isotretinoin; and b) at least one contraceptive agent (e.g. an estrogenic steroid, and/or a progestational steroid, and for example, wherein the contraceptive agent may be disposed in a second segment comprising a drug-permeable thermoplastic polymer.

In a different embodiment, a therapeutic device for delivery of an antimicrobial agent via the vagina is provided, comprising at least one segment comprising a drug permeable ethylene-vinyl acetate copolymer and the antimicrobial agent. An antimicrobial agent may be antibacterial agent, an antifungal agent (e.g., clotrimazole, nystatin, fluconazole, ketoconazole, amphotericin B, capsofugin and voriconazole, or combinations thereof) or an anti-viral agent, for example, the antibacterial agent may chosen from metronidazole, tinidazole and combinations thereof. An anti-viral agent may be tenofovir, and the device may additionally include UC781. For example, a device may include a first antiviral agent (e.g. tenofovir) disposed in a first segment, and a second antiviral agent (e.g. UC781) disposed in a second segment. Alternatively, a device may include segment that is unitary segment having a substantially uniform composition comprising the antimicrobial agent and copolymer, and/or may further comprise a contraceptive agent.

Also provided herein is a therapeutic device for delivery of a pharmacologically active compound capable of treating a chronic disorder, comprising at least one segment comprising a drug permeable thermoplastic polymer and the pharmacologically active compound. Such pharmacologically active compound contemplated herein may be chosen from cholesterol lowering medications, beta blockers, nitroglycerin, calcium channel blockers, aspirin and combinations thereof, e.g., a statin, an ACE inhibitor or an angiotensin II receptor antagonist; bronchodilators, antibiotics, and combinations thereof. Alternatively or additionally, a contemplated device may include a pharmacologically active compound such as a anti-nausea drug or an analgesic.

In certain embodiments, a therapeutic device may be a vaginal ring, e.g. with an overall diameter of from 40 mm to 80 mm, and/or a cross-sectional diameter of from 0.5 mm to 12 mm. Alternatively, a contemplated device may further comprises a tampon associated with at least one segment.

Also provided, in certain embodiments, is a method of treating endometriosis, uterine fibroids, and/or breast cancer, comprising vaginally administering a pharmaceutically effective amount of leuprolide or a pharmaceutically acceptable salt thereof to a female patient in need thereof. Such methods may include administering vaginally administering leuprolide by e.g. a) positioning in the vaginal tract of the female patient the therapeutic device or vaginal ring as provided herein and b) maintaining the therapeutic device in the vaginal tract of the female patient for a period of time sufficient to deliver a pharmaceutically effective amount of the leuprolide to the female patient.

### DESCRIPTION OF DRAWINGS

FIG. 1A depicts a ring-shaped intravaginal therapeutic device 10, consisting of one segment 12.

FIG. 1B depicts a ring shaped intravaginal therapeutic device, consisting of segments 12 and 13, which are connected to each other by a coupling means 14.

FIG. 1C depicts a ring as described herein.

FIG. 2 depicts LH response (miU/ml) in subjects after placement of a therapeutic device including 18 mg leuprolide as disclosed herein.

FIG. 3 depicts FSH response (miU/ml) in subjects after placement of a therapeutic device including leuprolide as disclosed herein.

FIG. 4 depicts estradiol response (pg/ml) in subjects after placement of a therapeutic device including leuprolide as disclosed herein.

FIG. 5 depicts progesterone response (ng/ml) in subjects after placement of a therapeutic device including leuprolide as disclosed herein.

FIG. 6 depicts results of mass spectra analysis in a subject after placement of a therapeutic device including leuprolide as disclosed herein.

FIG. 7 depicts results of a comparison of serum leuprolide levels in a patient administered Lupron® depot (22.5 mg) by injection and patients administered leuprolide vaginally through a ring formed from a unitary segment including a low dose (18 mg leuprolide) and ethylene vinyl acetate or a high dose (36 mg leuprolide) and ethylene vinyl acetate.

FIG. 8 compares LH response (miU/ml) in subjects after placement of a therapeutic device that includes low dose (18 mg) or high dose (36 mg) of leuprolide as disclosed herein.

FIG. 9 compares FSH response (miU/ml) in subjects after placement of a therapeutic device that includes low dose (18 mg) or high dose (36 mg) of leuprolide as disclosed herein.

FIG. 10 depicts estradiol response (pg/ml) in subjects after placement of a therapeutic device that includes low dose (18 mg) or high dose (36 mg) of leuprolide as disclosed herein.

FIG. 11 depicts progesterone response (ng/ml) in subjects after placement of a therapeutic device that includes low dose (18 mg) or high dose (36 mg) of leuprolide as disclosed herein.

FIG. 12 depicts a schematic diagram of a drug delivery system including a tampon shaped holder that includes material suitable for tampon use, and segmented EVA rods that include one or more drugs disposed on the tampon shaped holder.

FIG. 13 depicts a schematic diagram of a drug delivery device including a tampon shaped holder that includes material suitable for tampon use, and segmented EVA rods that include one or more drugs disposed within the tampon shaped holder; FIG. 13A depicts a device with the EVA rods disposed parallel to a longer axis of the tampon; FIG. 13B depicts a device with EVA rods disposed parallel to a shorter axis of the tampon or at angle to both axes of the tampon.

FIG. 14 depicts a segmented EVA rod containing three different segments each loaded with a different drug (A, B, or C) or with no drug (placebo).

FIG. 15 depicts the in vitro release kinetics intravaginal ring that includes leuprolide as described herein.

### DETAILED DESCRIPTION

The present invention relates in general to a therapeutic vaginal device for the delivery of therapeutic agents, and a method for the delivery of therapeutic agents to a female mammal. Contemplated drug delivery device may be useful for delivering one, two, or more drugs, and may include a tampon or tampon-like object, or may be ring shaped. The present invention also relates to methods for the treatment of obesity, methods for the treatment of diabetes, methods for the treatment of multiple sclerosis (MS), methods for the treatment of acne, methods for the treatment of hirsutism, methods for the treatment of bacterial vaginosis, methods for the prophylactic prevention of HIV, methods for the treatment of endometriosis, methods for the treatment of uterine fibroids, methods for the treatment of polycystic ovarian disease, methods for the treatment of breast cancer, and methods for the treatment of chronic diseases.

For convenience, before further description, the meaning of certain terms and phrases used in the specification, examples, and appended claims are provided below.

The term “therapeutic agents”, “active agents” or “drugs” refers to physiologically or pharmacologically active agents that produce a local and/or systemic effect in a mammal, such as a human, and are used for the purpose of disease therapy.

As used herein, “peptide” refers to short polymers formed from the linking, in a defined order, of amino acids. Such peptides include those natural and synthetic peptides having about 3 amino acids in length, e.g. about 4 amino acid, to about 40 peptides in length, e.g. about 28, 29, 36, or 37 amino acids. Exemplary peptides include ghrelin (about 28 amino acids), oxyntomodulin (37 amino acids), glatiramer acetate (about 4 amino acids), and leuprolide, and their pharmaceutically acceptable salts thereof.

As used herein, “therapeutic peptides” refer to naturally derived or synthetic peptides used for the purpose of disease therapy.

As used herein, the term “contraceptive agents” refer to one or more hormonal steroids (e.g., estrogenic steroids and/or progestational steroids) that prevent or reduce the likelihood of pregnancy.

The terms “estrogenic steroid” and “estrogen” are used interchangeably to refer to an agent, natural or synthetic, that exerts biological effects characteristic of estrogenic hormones such as estradiol. As used herein, the terms “estrogenic steroid” and “estrogen” also encompasses “conjugated estrogens,” which are an amorphous preparation of naturally occurring, water-soluble, conjugated forms of mixed estrogens that typically are obtained from the urine of pregnant mares (e.g., sodium estrone sulfate). Also included are “esterified estrogens,” which are a mixture of the sodium salts of sulfate esters or glucanoride of sulfate conjugates of estrogenic substances. Examples of suitable estrogens include, without limitation, estradiol valerate, estradiol benzoate, 17-βestradiol, estradiol cypionate, estrone, piperazine estrone sulfate, estriol, ethyl estradiol, polyestradiol phosphate, estrone potassium sulfate, benzestrol, chlorotrianisene, methallenestril, dienestrol, diethylstilbestrol diphosphate, mestranol, diethylstilbestrol (DES), quinestranol, phytoestrogens, animal-derived estrogens (e.g., equine estrogens), and metabolic derivatives of animal-derived estrogens. These also include any steroid or non-steroidal compound that binds either to the known estrogen receptors that exist within cells or to estrogen receptors that bind to extracellular membranes and cause biologic effects that mimic those of estradiol or other estrogenic compounds.

The terms “progestational steroid” and “progestin” are used interchangeably to refer to an agent, natural or synthetic, that affects some or all of the biological changes produced by progesterone, which is a hormone of the corpus luteum. For example, a progestin can induce secretory changes in the endometrium. Examples of progestins include, without limitation, progesterone, 17-hydroxy progesterone derivatives, 19-nor-testosterone derivatives, 19-nor-progesterone derivatives norethindrone, norethindrone acetate, norethynodrel, norgestrel, norgestimate, ethynodiol diacetate, allylestrenol, lynoestrenol, fuingestanol acetate, medrogestone, norgestrienone, dimethiderome, ethisterone, cyproterone levo-norgestrel, dl-norgestrel, cyproterone acetate, gestodene, desogestrol, dydrogesterone, ethynodiol diacetate, medroxyprogesterone acetate, megestrol acetate, phytoprogestins, animal-derived progestins, and metabolic derivatives of animal-derived progestins. These compounds also include any steroidal or non-steroidal compounds that bind to the cytoplasmic or membrane bound progesterone and mimic any of the biologic effects of progesterone or progestins.

As used herein, the term “antiandrogen” or “androgen antagonists” refers to any of a group of hormone receptor antagonists compounds that are capable of preventing or inhibiting the biologic effects of androgens on normally responsive tissues in the body.

The term “antimicrobial agent” generally refers to physiological or pharmacologically active agents that include antibacterial agents (e.g. antibiotics), antiviral agents, antifungals, and antiprotozoals.

As used herein, the term “antibacterial agent” refers to physiological or pharmacologically active agents that destroy bacteria or suppresses their growth or their ability to reproduce.

As used herein, the term “antiviral agent” refers to physiological or pharmacologically active agents that destroy viruses or suppresses their growth or their ability to reproduce.

As used herein, the terms “unitary segment” or “segment” refer to a solid material having a substantially uniform or homogenous composition throughout. The terms “segment” and “unitary segment” specifically exclude, in some embodiments, vaginal rings or portions thereof, segments, or forms that comprise a core or reservoir and an inner and/or outer layer of material, such as a skin, wall, membrane, coating, or polymeric layer or layers. The term “polymeric form” refers to solid material having a substantially uniform or homogenous composition throughout, and comprising a polymer. Such a form may have any shape, such as, e.g. a cylinder, rod, ovoid, string, thread, etc. In some embodiments, a unitary segment may consist essentially of a polymer (or polymer mixture) and/or a polymer (or polymer mixture) and a therapeutic agent (and optionally another therapeutic agent an/or pharmaceutically acceptable excipient).

As used herein, the terms “unitary cylindrical segment” and “unitary cylindrical rod” refer to a solid cylinder or rod-shaped material having a substantially uniform or homogenous composition throughout.

As used herein, the terms “drug-permeable”, “peptide-permeable”, or “agent-permeable” refer to a polymeric material through which a drug, a peptide can diffuse and thus be absorbed for local and/or systemic effects in a mammal.

The term “compatible” means compatible both with the environment of the vaginal tract in that there is no breakdown of the tensile nature or structural integrity of the device due to the contents of the vagina. Likewise there is no deleterious action on the sensitive tissue in the area of placement in the vaginal tract. Widely varying types of polymeric material are suitable in providing these non-toxic, drug-permeable properties, for example polysiloxanes, polyurethane, polyethylene, ethylene-vinyl acetate copolymers, cellulose, copolymers of polystyrene, polyacrylates and various types of polyamides and polyesters. The above-mentioned polymers can be used in a porous or microporous form.

The term “thermoplastic polymer” refers to a polymeric material which is capable of being softened by heating and hardened by cooling through a temperature range characteristic of the polymer, and in the softened state can be shaped by flow into devices by molding or extrusion.

As used herein, the term “coupling means” refers to a method, mechanism, material or device for joining or connecting the ends of two unitary segments or unitary cylindrical rods to each other. The term “adhesive material,” as used herein refers to an inert bonding agent, glue, or other substance having sufficient adhesive properties to bind the ends of the segments. The adhesive material can be, for example, a medical grade silicone adhesive.

As used herein, the terms “patient” and “female mammal” are used interchangeably to refer to a human or other animal in which it is desired to provide a medical treatment or contraceptive agent.

The term “tampon” refers to any type of absorbent structure that can be inserted into the vaginal cavity or other body cavities for the absorption of fluid from or for the delivery of active materials, such as medicaments or moisture. A tampon can be straight or non-linear in shape, such as curved along the longitudinal axis.

Generally, there are two types of tampons. The first type of tampon is a self-sustaining tampon. Tampons are generally “self-sustaining” in that they will tend to retain their general shape and size before use. A typical self-sustaining tampon is 35-60 mm long, the length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 mm wide corresponding to the largest cylindrical cross section. The width can vary along the length of the self-sustaining tampon.

The second type of tampon is an easily “deformable, fluid-permeable bag tampon”. The deformable, fluid-permeable bag tampon consists of, but is not limited to, pieces such as absorbent chips, spheres, or fibers such that the fluid permeable bag tampon is readily deformable with a force of less than about 3 psi. The tampon is substantially deformable at pressures of less than about 1 psi; resulting in the tampon spreading or being easily indented when the pressure is applied from a surface of about 0.15 mm diameter.

As used herein, the terms “vaginal cavity” and “within the vagina” refer to the internal genitalia of the human female in the pudendal region of the body. In the present specification, the term “intra-vaginal” includes both local delivery to the vagina as well as through the vagina to other target tissues in the body, including systemic effects.

As used herein, “pharmacologically effective amount,” “therapeutically effective amount” or simply “effective amount” refers to that amount of a drug effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically-acceptable salts” is art-recognized and refers to the relatively non-toxic, inorganic and organic acid addition salts of peptides. For example, salts of leuprolide include hydrochloric, hydrobromic, hydrofluoric, sulfuric, phosphoric, acetic, trifluoroacetic, maleic, succinic or methanesulfonic salts. Cationic salts are contemplated and may for example be prepared by treating a compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are some non-limiting examples of cations present in pharmaceutically acceptable salts.

### Systems and Devices

A therapeutic system or device for systemic and/or vaginal delivery of active agents as contemplated herein includes at least one polymeric form, e.g. a substantially cylindrical rod, string, or thread, that includes at least one, two, three or more segments, with a least one segment including a drug permeable thermoplastic polymer and an active agent. The device releases the drug over time when placed in the vagina of a patient. A segment may have a substantially uniform composition (e.g. of both the drug and polymer) throughout, and may be capable of releasing the drug over time when placed in the vaginal cavity of a patient. For example, this disclosure contemplates devices capable of delivering a pharmaceutically effective amount of one or more contraceptive agents intravaginally for about 1 day or more, about 1 week, about 1 month, about 3 month, or about 6 months or more, with or without replacing the device once placed within the vagina.

Contemplated therapeutic systems may include one or more segments which each may include the same active agent or each may include a different active agent. Each segment can optionally include further active agents, or, in the case of a device that includes two or more segments, different segments may each include a different drug, or one or more segments can include a drug and/or another therapeutic agent or an agent that augments delivery of an active agent, or one or more segments can include an active agent with another segment including another agent, or two or more segments may include the same active agent (e.g. in the same weight percentage or a different weight percentage), or one, two, or more segments may include no active agent. Similarly, a contemplated device that includes two or more segments may include a first segment with a different thermoplastic polymer than a second segment. For example, a first segment may include a thermoplastic polymer with a different release rate than a second segment (which may result from e.g. a different polymer or a different percentage of monomer, e.g. a different percentage of vinyl acetate in ethylene vinyl acetate co-polymer.)

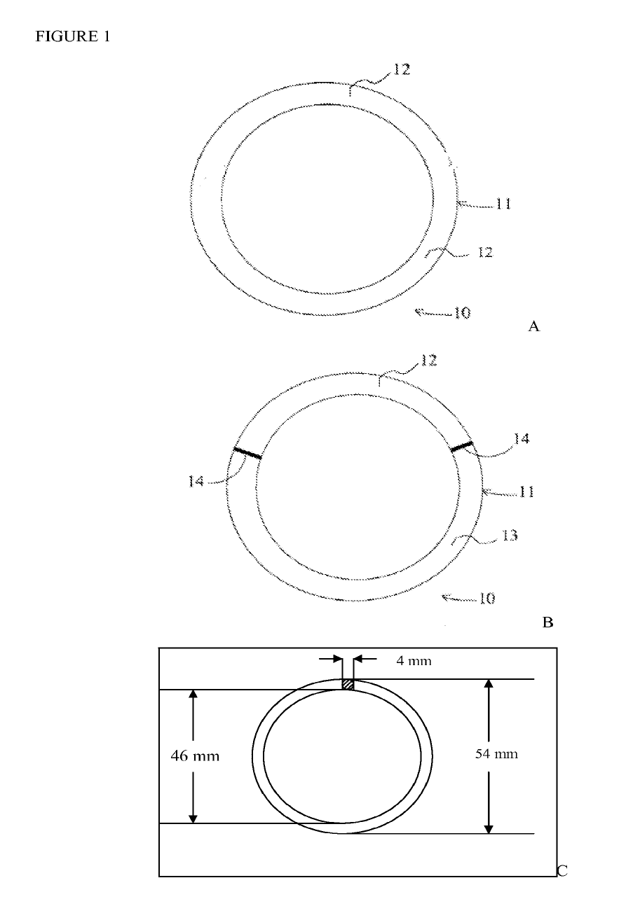

In an embodiment, a unitary segment may be formed in a ring shape. In another embodiment, two, three or more segments may be joined end to end to form a ring shape. For example, at least one end of a segment may be attached to the end of another unitary segment by a coupling means, such as an adhesive material or by annealing the ends of the segments to same or different thermoplastic polymers. In an exemplary embodiment, a drug delivery device may include the device depicted in FIG. 1A, wherein the drug delivery device 10 comprises a body 11 sized, shaped and adapted for placement in the vaginal tract of a human. Body 11 may be formed of a polymer that releases a drug(s) by diffusion into the vaginal tract of the patient. Drug delivery device 10 may include two unitary cylindrical segments 12 and 13 as depicted in FIG. 1B which are connected to each other by a coupling means 14. The two segments can also be directly fused without the need for a coupling means, or alternatively, the ring may be formed from one segment, as e.g., shown in FIG. 1A, which may eliminate the need for coupling means 14. Although the illustrated device comprises one or two segments, the drug delivery device of the present invention can comprise one, three, four, five, six, or more segments. The number and size of the segments used for a particular application will depend, inter alia, on the number of drugs to be delivered, the dosages of the drugs, and the need for a placebo segment(s) to prevent diffusion and interaction of the drugs within the device. For example, a contemplated ring may consist essentially of an unitary segment that includes, or in some embodiments, consists essentially of ethylene vinyl acetate copolymer and/or polyethylene glycol, and an effective amount of therapeutic peptide and optionally, a pharmaceutically acceptable excipient.

In some embodiments, contemplated systems or devices that include e.g. EVA and/or PEG and a therapeutic peptide, and optionally an excipient such as a surfactant and/or an emulsifier such as a nonionic surfactant, e.g., Tween (for example Tween 80 or polysorbate 80) may be a stand alone implantable body having a homogenous cross-section at all points along a length of the implantable body. The disclosed system, devices, segments, and rings, for example, may, in some embodiments, have a cross-sectional diameter substantially identical to a cross-sectional diameter of the implantable body, e.g. at all points along a length (e.g. an entire ring).

Contemplated therapeutic systems or devices may comprise a polymeric form that includes a drug and a tampon or tampon-like structure, for example, a cylinder made of absorbing materials such as cotton or super absorbent hydrogel and a thread or a string or a rod made of e.g. segmented EVA and/or PEG and e.g. a therapeutic peptide. For example, contemplated herein is a polymeric shape, e.g., a substantially cylindrical rod, thread, or string that includes one, two, three, or more segments of a thermoplastic polymer with at least one segment including an active agent, wherein the shape is imbedded inside the cylinder of absorbing material, or attached to the surface of e.g. a tampon for delivery of a therapeutic or active agent. In another embodiment, a segmented thread or a string or a rod, including EVA, is imbedded or attached to the surface of an absorbing cylinder. The delivery system of the invention is composed of a tampon-like support, such as cotton or a non-woven polymer material. For example, a cylinder made of EVA is inserted into the support as a rod, or as strands or as a string. The EVA cylinder along its shorter axis has cross section which is homogenous and along its longer axes it is segmented. The EVA cylinder could be placed near the surface of the tampon or inside the tampon. For example, an EVA delivery system is positioned between the inner absorbent core of the tampon and its outer covering layer. The EVA/drug combination may be in a substantially cylindrical form or shape, although other shapes are also possible. A plurality of these polymeric shapes, e.g. rods, may be placed on different sides of the tampon in order to release the drug in all directions. Alternatively, the drug delivery device may include one or more segments or polymeric shapes in the shape of a ring, a wafer, or a suppository.

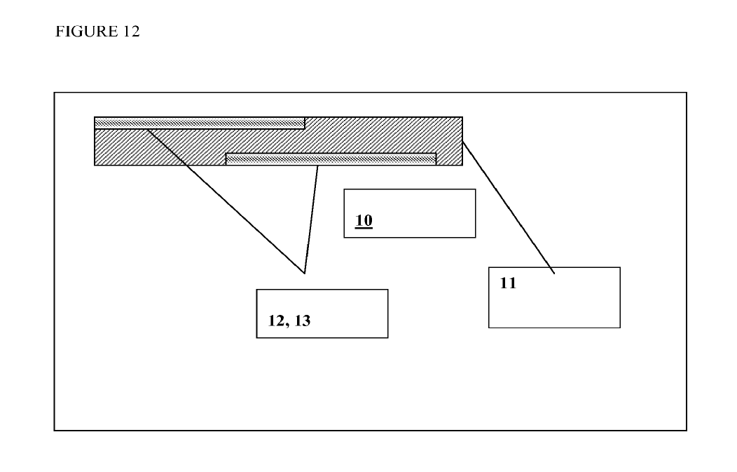

By way of example, FIG. 12 shows one embodiment of the present invention. The drug delivery device shown in this and other figures is offered for illustration only, and is not to be construed as limiting the invention. As one of skill in the art will appreciate, the drug delivery device can be manufactured in a variety of shapes, sizes, and dimensions, depending upon the particular mammal to be treated, as well as the nature and severity of the condition to be treated.

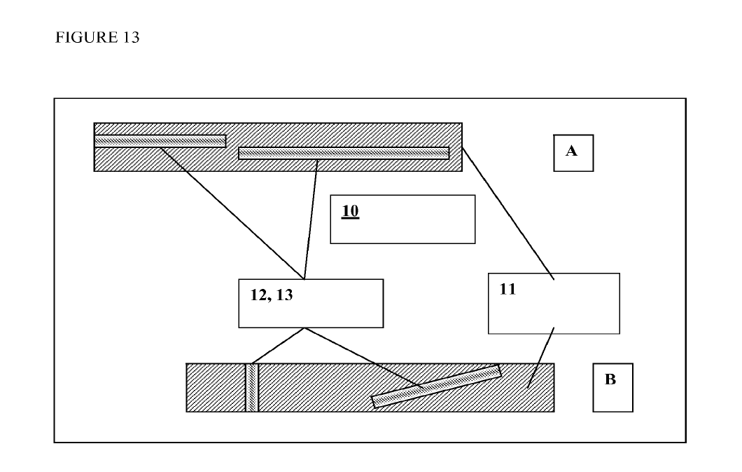

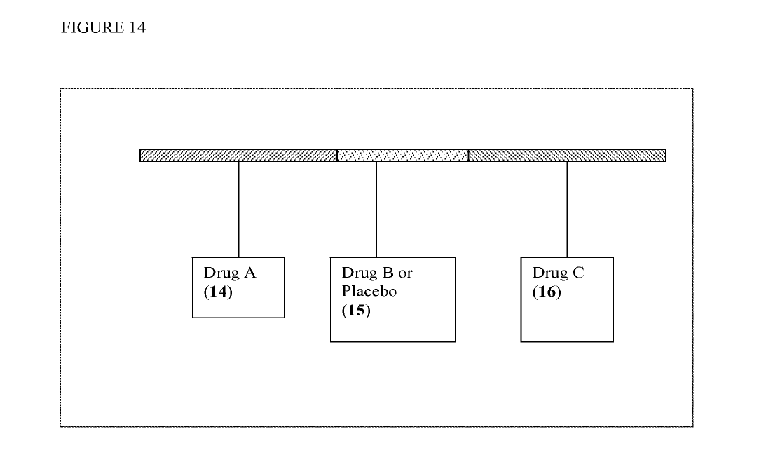

FIG. 12 depicts a drug delivery device 10 comprising a tampon 11 sized, shaped and adapted for placement in the vaginal tract of a human. The drug delivery device 10 further comprises two segmented EVA rods 12, 13 formed of a polymer that releases a drug(s) by diffusion into the vaginal tract of the patient. Rods 12, 13 of segmented EVA shown in FIG. 12 can comprises three unitary cylindrical segments 14, 15, 16, as shown in FIG. 14, and can be disposed substantially parallel to the long axis of the tampon (FIG. 12 or 13A) or substantially perpendicular or at an other angle to the long axis of the tampon. (FIG. 13B). FIG. 12 depicts rods 12, 13 disposed on the surface of tampon 11, while FIG. 13 depicts rods 12, 13 disposed with the tampon 11. Although the figures depict unitary cylindrical segments one of skill in the art will appreciate the segments can be manufactured in a variety of shapes, sizes, and dimensions. The two segments can also be directly fused without the need for a coupling means. The number and size of the segments used for a particular application will depend, inter alia, on the number of drugs to be delivered the dosages of the drugs, and the need for a placebo segment(s) to prevent diffusion and interaction of the drugs within the device.

For example, a contemplated delivery system may include two or more unitary segments, wherein at least one of the segments comprises a uniform mixture of a drug-permeable polymeric substance (e.g. EVA or a combination of EVA and PEG, and optionally an excipient such as Tween 80) and a first active agent, and a second segment includes a second drug-permeable polymeric substance and a second active agent, with an optional third segment which may include another active agent which may be the same or different than that in the second segment. At least two of the segments may comprise a different active agent. In some embodiments, the first and second permeable polymeric substance may be the same, e.g., a thermoplastic polymer, such as an ethylene-vinyl acetate copolymer. When the drug delivery system includes one or more polymeric shapes for the release of two active agents (e.g. both an antiandrogen and a contraceptive agent, or isotretinoin and a contraceptive agent) the system may release each agent in a substantially constant ratio over a prolonged period of time.

Contemplated therapeutic devices include a vaginal ring that includes ethylene-vinyl-acetate co-polymer and an amount of an active agent appropriate for systemic delivery over time to patient when placed in the vagina. In some embodiments, a vaginal ring is contemplated that is formed from one unitary segment, and for example, consists essentially of ethylene vinyl co-polymer and an active agent and optionally one or more pharmaceutically acceptable excipients. Such a ring may be capable of delivering an active agent to a patient with a reduced and/or delayed peak serum concentration, for example, as compared to a patient administered the active agent as a depot injection (for example Lupron Depot® having 22.5 mg, 30 mg, 7.5 mg, 11.25 mg, and/or 3.75 mg of leuprolide acetate in a polylactic acid depot, or depot composition comprises about 11.25 mg or 22.5 mg of leuprolide acetate and polylactic acid).

In an embodiment, the disclosed devices e.g. rings may be capable of delivering an active agent to a patient with a decrease in peak serum concentration, for example, as compared to a patient administered the active agent as a depot injection, but may deliver appropriate amounts of the active agent effective to achieve a treatment over 3 days, 1 week, 1 month, or more. Such an exemplary vaginal ring, that effectively includes an active agent and ethylene vinyl co-polymer (and optionally a pharmaceutically acceptable excipient) can deliver systemically, in some embodiments, a non-linear increase in the amount of active agent to a patient, with respect to the amount of active agent present in the ring, which, in some embodiments, may not be achievable if other active agents and/or polymers and/or peptides are present in the device.

For example, contemplated herein are therapeutic devices including unitary segments that include ethylene-vinyl-acetate co-polymer and an active agent wherein an increase in dose of active agent present in the ring results in a greater increase of peak serum agent in a patient than the expected serum level resulting from the increased dosage in the device. For example, a doubling of the dose of active agent in the device leads to an about three-fold increase in peak serum levels of the active agent in patient (once placed in the vagina of the patient).

In one embodiment, systemic administration using a vaginal device, e.g. a ring, may result in a peak serum concentration of the active agent (e.g. leuprolide) in a patient at about 12 to about 22 hours, e.g. about 14 to about 17 hours, about 15 or about 16 hours after insertion of the device. In another embodiment, contemplated herein are devices that include about 18 mg to about 100 mg of therapeutic leuprolide, e.g. about 18 mg, about 36 mg, or about 54 mg or more. For example, upon administration, disclosed devices, once inserted in a patient, may result in a serum level of e.g. leuprolide in the patient, of about 0.01 ng/mL to about 2.0 ng/mL, or about 0.1 ng/mL to about 1.0 ng/mL, e.g. about 0.6 ng/mL or about 1.0 ng/mL, after about 12 hours, after about 18 hours, after about 20 hours, or even after about 1 day. Exemplary peak leuprolide levels using a disclosed device in a patient may be about 0.5 ng/mL to about 4 ng/mL, at for example, about 16 hours after patient insertion.

In order to achieve constant levels of each of one or more active agents and avoid the inefficiencies of concentration peaks and valleys, active agents may be released from a delivery system at a rate that does not substantially change with time (so called zero-order release). Preferably, the initial dose of an active agent is the therapeutic dose, which is maintained by the delivery system.

In an embodiment, a disclosed device provides for substantially “zero order kinetic” active agent administration, in which an active agent is released in a steady state, thus providing a corresponding predictable absorption and metabolism of the active agent in the body tissues. For example, contemplated therapeutic devices that include leuprolide, upon insertion into a patient's (e.g. a human) vagina, may result in a peak serum concentration of leuprolide about 12 to about 22 hours, e.g. about 15, 16, or 17 hours after insertion. Such a peak serum concentration in a patient may be less than that of a patient administered a leuprolide depot concentration by injection (such as a depot composition having 22.5 mg or 11.5 mg of leuprolide, e.g. Lupron® depot). For example, after insertion of a disclosed device that includes leuprolide, a patient may have peak serum levels of FSH and/or LH about 12 to about 18 hours after insertion, e.g. at about 15 or about 16 hours. Such peak levels of FSH and LH may occur later in a patient as compared to the time peak levels of FSH and LH occur in a patient administered a depot composition of leuprolide (such as Lupron Depot®). In some embodiments, a contemplated therapeutic device may release about 5 μg to about 150 μg/day, e.g. about 10 μg/day of a therapeutic protein, e.g. leuprolide, upon insertion into the vagina of a patient.

In one embodiment, the delivery of active agents may be “targeted” to the specific body organ, where the intended therapeutic effect is desired; other organs such as liver, in which unintended effects may occur, may be bypassed. Thus, the efficient metabolic and therapeutic use of one or more active agents may be enhanced, and the development of adverse metabolic side effects may be reduced.

### Active Agents

The one, two, three, or more active agents used in the delivery system of the invention may be any agent which is released and acts locally or which is absorbed through the vaginal mucosa to other locations in the body and acts systemically. Any pharmaceutically active agent used to treat the body, and which is capable of diffusing through the polymer and being absorbed by the lining of the vaginal tract, is useful in the present invention. The active agent, e.g., may be present in the device or system in combination with a biocompatible excipient or carrier acceptable for application of the active agent to the vaginal epithelium. Although the mechanism may be diffusion-controlled, the co-inclusion of excipients such as wetting agents or surfactants in the formulation may be necessary. For example, in an embodiment, isotretinoin or antiandrogens of the disclosed device is absorbable through the vaginal mucosa and thereby transmitted via venous and lymphatic channels to the uterus or to the general blood circulation.

Therapeutic peptides contemplated herein include peptides about 3 amino acids in length to about 50 amino acids in length, e.g. about 4 to about 10 amino acids in length, about 4 to about 25 amino acids in length, or about 25 to about 40 amino acids in length. Exemplary peptides include GLP-1 and its synthetic derivatives or analogs thereof such as exenatide, liraglutide, taspoglutide, and albiglutide, GIP and its synthetic derivatives or analogs thereof, ghrelin and its synthetic derivatives or analogs thereof such as RC-1139 and BIM-28163, GLP-2 and its synthetic derivatives or analogs thereof, oxyntomodulin and its synthetic derivatives or analogs thereof such as TKS1225, peptide YY and its synthetic derivatives or analogs thereof such as AC162352, pancreatic polypeptide (PP) and its synthetic derivatives or analogs thereof such as TM30339, leptin and its synthetic derivatives or analogs thereof, and amylin and its synthetic derivatives or analogs thereof such as pramlintide. Therapeutic peptides contemplated herein may also include luteinizing hormone releasing hormone (LHRH) agonists such as leuprolide (p-Glu-His-Trp-Ser-Tyr-D-Leu-Leu-Arg-Pro-NHEt) or pharmaceutically acceptable salts thereof, e.g. leuprolide acetate. Other contemplated therapeutic peptides include glatiramer acetate, goserelin acetate, salmon calcitonin, glatiramer acetate, octerotide acetate, desmopressin, bivalirudin, eptifibatide, enfuvirtide, abarelix, and zadaxin.

Other therapeutic agents contemplated herein include a DPP-IV inhibitors or its synthetic derivatives such as sitagliptin, vildagliptin, alogliptin, saxagliptin. Therapeutic agents that may be administered via the disclosed device may also include biguanides such as metformin, pioglitazone, sulfonylureas, and insulin.

Anti-androgens contemplated for use in the disclosed devices include spironolactone, a synthetic 17-spirolactone corticosteroid, cyproterone acetate, flutamide, nilutamide, bicalutamide, ketoconazole, finasteride, and dutasteride. In an exemplary embodiment, a device is provided that includes spironolactone in a segment of the device, and one or more contraceptive agents in one or more further segments of the device.

Contraceptive agents contemplated for use in the disclosed device include progestins, e.g., etonogestrel, and estrogens (e.g., ethinyl estradiol). For example, a contemplated device may include a first segment comprising an antiandrogen or isotretinoin, and a second segment including a progestin, and/or a third segment that includes an estrogen. An alternative exemplary device may include a first segment that includes an antiandrogen or isotretinoin and a first contraceptive agent, and a different segment that includes a second contraceptive agent.

Antibacterial agents contemplated herein include metronidazole, tinidazole, clindamycin, and sulfamides. Antiviral agents contemplated herein include tenofovir, UC781, and the like. Antifungal agents include clotrimazole, nystatin, fluconzazole, ketoconazole, amphotericin B, caspofugin, voriconazole, and the like. In an embodiment, contemplated agents may be substantially water soluble and/or may be absorbed by tissues. For example, tenofovir is water soluble and may be absorbed by tissues.

Contemplated agents for the treatment of coronary heart disease include, for example, one or more of cholesterol lowering medications, beta-blockers, nitroglycerin, calcium channel blockers, aspirin, and combinations thereof. In one embodiment, a cholesterol lowering medication is a statin or an Angiotensin-Converting Enzyme (ACE) inhibitor. For example, statins contemplated for use in the disclosed device include, but are not limited to, atorvastatin, fluvastatin, itavastatin, lovastatin, pravastatin, rosuvastatin, and simvastatin. ACE inhibitors contemplated for use in the disclosed device include, but are not limited to, captopril, zofenopril, enalapril, ramipril, quinapril, perindopril, lisinopril, benazepril, and fosinopril. Beta blockers contemplated for use in the disclosed device include, but are not limited to, alprenolol, carteolol, levobunolol, mepindolol, metipranolol, nadolol, oxprenolol, penbutolol, pindolol, propranolol, sotalol, timolol, acebutolol, atenolol, betaxolol, bisoprolol, esmolol, metoprolol, nebivolol, amosulalol, landiolol, tilisolol, arotinolol, carvedilol, celiprolol, labetalol, and butaxamine. Calcium channel blockers contemplated for use in the disclosed device include, but are not limited to, amlodipine, aranidipine, azelnidipine, barnidipine, benidipine, cilnidipine, clevidipine, efonidipine, felodipine, lacidipine, lercanidipine, manidipine, nicardipine, nifedipine, nilvadipine, nimodipine, nisoldipine, nitrendipine, pranidipine, verapamil, gallopamil, and diltiazem.

The present disclosure contemplates the use of COPD and/or asthma treatment agents in a disclosed device, e.g. one or more of bronchodilators, antibiotics, and combinations thereof. Bronchodilators contemplated for use in the disclosed device include, but are not limited to, (1) β2 agonists, such as, pirbuterol, ephedrine, albuterol, salmeterol, levalbuterol, bambuterol, formoterol, clenbuterol, (2) anticholinergics, such as, ipratropium, tiotropium, (3) cromones, such as cromoglicic acid and nedocromil sodium, (4) leukotriene antagonists, such as, montelukast, pranlukast, and zafirlukast, and (5) xanthines such as theophylline. Antibiotics contemplated for use in the disclosed device include, but are not limited to, doxycycline, trimethoprim-sulfamethoxazole, amoxicillin-clavulanate potassium, penicillins, fluoroquinolones, cephalosporins, and aminoglycosides. For example, the device can include albuterol in a first segment of the device and doxycycline in a second segment of the device.

In another embodiment, the present disclosure contemplates a device that includes a chronic kidney disease treatment agent, such as one or more of: ACE inhibitors, angiotensin II receptor antagonists, or a combination thereof. ACE inhibitors contemplated for use in the disclosed device include, but are not limited to, captopril, zofenopril, enalapril, ramipril, quinapril, perindopril, lisinopril, benazepril, and fosinopril. Angiotensin II receptor antagonists contemplated for use in the disclosed device include, but are not limited to, valsartan, telmisartan, losartan, and irbesartan. For example, the device can include captopril in first segment of the device and valsartan in a second segment of the device.

In a further embodiment, the present disclosure contemplates a device that includes one or more of: an anti-migraine drug, an anti-nausea drug, an analgesic or a combination thereof for, e.g., the treatment of migraine. Anti-nausea drugs contemplated for use in the disclosed device include, but are not limited to, phochlorperazine, promethazine hydrochloride, metoclopramide hydrochloride, trimethobenzamide hydrochloride, and ondansetron hydrochloride. Analgesics contemplated for use in the disclosed device include, but are not limited to, acetaminophen, non-steroidal anti-inflammatory drugs, such as aspirin, amoxiprin, benorylate, choline magnesium salicylate, diflunisal, ethenzamide, faislamine, methyl salicylate, magnesium salicylate, salicyl salicylate, salicylamide, diclofenac, aceclofenac, acemethacin, alclofenac, bromfenac, etodolac, indometacin, nabumetone, oxametacin, proglumetacin, sulindac, tolmetrin, and ibuprofen, alminoprofen, benoxaprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, sulfinpyrazone, piroxicam, droxicam, lornoxicam, meloxicam, tenoxicam, and COX-2 inhibitors. Other contemplated agents include ergotamine, ergostine, butalbital, Phenobarbital, acetaminophen, diclofenac sodium, theadone, sumatriptan, naratriptan, razatriptan, zolmitriptan, almotriptan, eletriptan, gabapetin, and the like. For example, the device can include phochlorperazine in a first segment of the device and one of the non-steroidal anti-inflammatory agents in a second segment of the device.

In some embodiments, active agents contemplated herein includes a non hormonal and/or non steroidal compound. In an embodiment, a contemplated device or method may not include a contraceptive agent or hormone, e.g. an estrogenic or progestation steroid.

Other examples of suitable active drugs include, without limitation, interferon, anti-angiogenesis factors, antibodies, antigens, polysaccharides, growth factors, hormones including insulin, glucogen, parathyroid and pituitary hormones, calcitonin, vasopressin renin, prolactin, thyroid stimulating hormone, corticotrophin, follicle stimulating hormone, luteinizing hormone and chorionic gonadotropins; enzymes including soybean, tyrpsin inhibitor, lysozyme, catalase, tumor angiogenesis factor, cartilage factor, transferases, hydrolases, lysases, isomerases, proteases, ligases and oxidoreductases such as esterases, phosphatases, glysidases, and peptidases; enzyme inhibitors such as leupeptin, antipain, chrymostatin and pepstatin; and drugs such as steroids, anti-cancer drugs or antibiotics. Suitable pharmaceuticals for parenteral administration are well known as is exemplified by the Handbook on Injectable Drugs, 6th edition, by Lawrence A. Trissel, American Society of Hospital Pharmacists, Bethesda, Md., 1990 (hereby incorporated by reference).

Additional examples of drugs which may be delivered by drug delivery devices according to this invention include, without limitation, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17 hydroxyprogesterone acetate, 19-nor-progesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, capropril, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, and imipramine. In some embodiments, the drug(s) to be delivered has a molecular weight of between 50 and 2000, more preferably between 200 and 1300.

In some embodiments, one or more drugs can be present within a contemplated device, and may include one or more of: a hormone replacement steroid or a contraceptive agent, for example an estrogenic compound, a progestational compound, and/or a gonadotropin releasing hormone or its peptide or non-peptide agonists or antagonist analogues, an interferon, anti-angiogenesis factors, growth factors, hormones, enzymes, transferases, hydrolases, lysases, isomerases, proteases, ligases and oxidoreductases, enzyme inhibitors, steroids, anti-cancer drugs, antibiotics, growth hormone, polysaccharides, antigens, and antibodies.

The amount of therapeutic agents incorporated in the drug delivery device varies depending on the particular agent the desired therapeutic effect, and the time span for which the device provides therapy. Since the inventive device is intended to provide dosage regimes for therapy for a variety of applications and indications, there is no critical upper limit on the amount of agent incorporated in the device. Similarly, the lower limit will depend on the activity of the agent and the time span of its release from the device.

The dosage unit amount for conventional beneficial drugs as described herein is well known in the art (see, e.g., Remington's Pharmaceutical Science (Fourteenth ed., Part IV, Mack Publishing Co., Easton, Pa., 1970). The amount of drug incorporated in the drug delivery device varies depending on the particular drug, the desired therapeutic effect, and the time span for which the device provides therapy. Since the inventive device is intended to provide dosage regimes for therapy for a variety of applications and indications, there is no critical upper limit on the amount of drug incorporated in the device. Similarly, the lower limit will depend on the activity of the drug and the time span of its release from the device.

The relative amount(s) of the agents(s) to be released can be modified over a wide range depending upon the active agent to be administered or the desired effect. Generally, the agent can be present in an amount which will be released over controlled periods of time, according to predetermined desired rates, which rates are dependent upon the initial concentration of the active substance in the polymeric matrix. In one embodiment, a rate may also depend upon the level of ultrasonic energy to which it is subjected. This necessarily implies a quantity of active substance greater than the standard single dosage. Proportions suitable for the purposes of this invention can range from about 0.01 to 50 parts by weight of the active substance to between about 99.99 and about 50 parts by weight of the polymeric matrix, preferably between about 10 and about 30 parts by weight in the case of an active agent to be implanted to give 100 parts per weight of the final system. The polymeric matrix in the composition to be released can be admixed in any convenient manner, for example by mixing the components as powders and subsequently forming the mixture into a desired shape such as by thermal forming at a temperature less than that which the composition will become degraded and at which the polymer has desired morphological properties.

The drug may be present in the device or system in combination with a biocompatible excipient or carrier acceptable for application of the drug to the vaginal epithelium. Although the mechanism may be diffusion-controlled, the co-inclusion of excipients such as wetting agents or surfactants in the formulation may be necessary. Disclosed therapeutic devices may optionally include pharmaceutically acceptable excipients, such as polaxomers, carbomers, polyvinyl alcohol, silicon dioxide, sodium carboxymethyl cellulose and/or combinations thereof. Other pharmaceutically acceptable excipients include α-lipoic acid, α-tocopherol, ascorbyl palmitate, benzyl alcohol, biotin, bisulfites, boron, butylated hydroxyanisole, butylated hydroxytoluene, ascorbic acid, carotenoids, calcium citrate, acetyl-L-carnitine, chelating agents, chondroitin, chromium, citric acid, coenzyme Q-10, cysteine, cysteine hydrochloride, 3-dehydroshikimic acid, EDTA, ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol, tocopherol acetate, tocopherol succinate, tocotrienal, d-α-tocopherol acetate, vitamin A, vitamin B, vitamin C, vitamin D, vitamin E, zinc, carbohydrates and combinations thereof. For example, contemplated excipients may include one or more of sodium acetate, sodium carbonate, citrate, glycylglycine, histidine, glycine, lysine, arginin, sodium dihydrogen phosphate, disodium hydrogen phosphate, sodium phosphate, and tris(hydroxymethyl)-aminomethane, bicine, tricine, malic acid, succinate, maleic acid, fumaric acid, tartaric acid, citric acid, aspartic acid or ethylenediaminetetraacetic acid (EDTA). In an embodiment, disclosed therapeutic devices include one or more carbohydrates and/or citric acid and/or one or more cellulose ethers (such as hydroxypropyl methylcellulose). In an embodiment, a drug of the disclosed device is absorbable through the vaginal mucosa and thereby transmitted via venous and lymphatic channels to the uterus or to the general blood circulation.

### Polymers

In some embodiments, a drug-permeable polymer of a segment may comprise, for example, olefin and vinyl-type polymers, carbohydrate-type polymers, condensation-type polymers, rubber-type polymers, and organosilicon polymers. Other drug permeable polymers include without limitation, poly(ethylene-vinyl acetate), poly(methylacrylate), poly(butylmethacrylate), plasticized poly(vinylchloride), plasticized nylon, plasticized soft nylon, plasticized poly(ethylene terephthalate), poly(ethylene), poly(acrylonitrile), poly(trifluorochloroethylene), poly(4,4′-isopropylene-diphenylene carbonate), poly(ethylenevinyl esters), poly(vinyl chloridediethyl fumarate), poly(esters of acrylic and methacrylic), cellulose acetate, cellulose acylates, partially hydrolyzed poly(vinyl acetate), poly(vinyl butyral), poly(amides), poly(vinyl carbonate), poly(urethane), poly(olefins), and the like. These polymers and their physical properties are known to the art and can be synthesized according to the procedures disclosed, for example, in Encyclopedia of Polymer Science and Technology (Interscience Publishers, Inc., New York, 1971) Vol. 15, pp. 508-530; Polymers (1976), Vol. 17, 938-956; Technical Bulletin SCR-159, 1965, Shell Corp., New York; and references cited therein; and in Handbook of Common Polymers, Scott and Roff (CRC Press, Cleveland, Ohio, 1971).

In an embodiment, the drug permeable polymer is capable of being degraded by ultrasonic energy such that any incorporated agent is released at a rate within a desired release range, or, in the case of nondegradable polymers, release is enhanced. Representative suitable polymers for this embodiment include polyesters such as poly(lactic acid), poly(lactic-co-glycolic acid), and/or polyanhydrides having the formula described in U.S. Pat. No. 4,657,543 (Langer et al.), which is incorporated by reference in its entirety herein. The monomers in any copolymer can be distributed regularly or at random. For example, an anhydride linkage may be highly reactive toward hydrolysis, and therefore, in some embodiments, it may be preferable that the polymer backbone be hydrophobic in order to attain the heterogeneous erosion of the encapsulated composition.

Hydrophobicity of polymers can be regulated easily, for example, by regulating the concentration of aromatic moieties in the linking backbone, or by monitoring the monomer ratio in the copolymer. In one embodiment, a polymeric backbone comprises or is formed from an acid such as 1-phenylamine, tryptophan, tyrosine or glycine. Other contemplated polymers include ethylene-vinyl acetate, poly(lactic acid), poly(glutamic acid), polycaprolactone, lactic/glycolic acid copolymers, polyorthoesters, polyamides or the like. Non-degradable polymers include ethylene-vinyl acetate, silicone, hydrogels such as polyhydroxyethylmethacrylate, polyvinyl alcohol, and the like.

In addition to providing appropriate release properties, a drug permeable polymeric substance may be formed from a compatible, non-absorbable, non-toxic polymeric substance that does not significantly induce a significant tissue reaction at the site of placement in the vaginal tract of the female mammal.

In one embodiment, one more segments comprise ethylene-vinyl acetate (EVA) copolymer and/or polyethylene glycol (PEG). Suitable EVA polymers include, for example, the EVA material manufactured by Aldrich Chemical Co. (Cat. No. 34,050-2); Evatane®. with the designations 28-150, 28-399, and 28-400, supplied by ICI and 28.420, and in particular 28.25 and 33.25 supplied by Atochem; and Elvax® with the designations 310, 250, 230, 220, and 210, supplied by Du Pont de Nemours. Exemplary EVA polymers may include a mixture of EVA having a 27-29 weight percent vinyl acetate content and EVA having a 17-19 weight percent vinyl acetate content, e.g. Evatane®18-150 and 28-25.

One or more segments may, in some embodiments, also include PEG, such as a PEG with a weight average molecular weight of about 2000 Da to about 8000 Da, e.g., about 3600 Da to about 4400 Da (e.g. 4000 Da).

When a contemplated drug delivery device comprises EVA, drug release may be determined by the vinyl acetate content of polymeric substance. The present invention contemplates use of EVA copolymers having a vinyl acetate content of about 4 to 80% by weight of the total, and a melt index of about 0.1 to 1000 grams per ten minutes. Melt index is the number of grams of polymer which can be forced through a standard cylindrical orifice under a standard pressure at a standard temperature, and thus is inversely related to the molecular weight of the polymer. In some embodiments, the EVA has a vinyl acetate content of about 4 to 50% by weight and a melt index of about 0.5 to 250 grams per ten minutes. For example, a contemplated unitary segment includes about 40% weight percent vinyl acetate and/a melt index of about 48 to about 62 grams per ten minutes, e.g. 57 grams per ten minutes, at e.g. 190° C./2.16 kg. In some embodiments, the disclosed devices include Evatane® 40-55, described at www.arkema-inc.com/tds/1126.pdf, hereby incorporated by reference. In some embodiment, the amount of vinyl acetate present in a finally processed ring is minimal or substantially undetectable.

In general, however, the rate of passage of an active agent through the polymer can be dependent on the molecular weight and solubility of the agent therein, as well as on the vinyl acetate content of the polymer, and in some embodiments, selection of particular EVA compositions will depend on the particular active agent to be delivered. For example, by varying the composition and properties of the EVA, the dosage rate per area of the device can be controlled, for example, different segments of a polymeric shape can each include different compositions of EVA. Thus, devices of the same surface area can provide different dosage of an active agent by varying the characteristics of the EVA copolymer. The release of the active agent by a drug delivery device comprising EVA can also be controlled by the surface area of the segment. For example, the length and/or circumference of the segment can increased, in some embodiments, to increase the rate of release of the active agent.

In addition to varying the percentage of vinyl acetate in the copolymer and the melt index or molecular weight, the properties of the copolymer can be changed by selectively hydrolyzing its acetate groups to alcohol groups. By converting a portion of the vinyl acetate units of the polymer to vinyl alcohol units, the polymer can rendered more hydrophilic and the rate of passage of relatively hydrophilic active agents may be increased. The percentage of vinyl acetate units hydrolyzed to vinyl alcohol units can vary widely but typically from about 20 to 60% are converted. This partial hydrolysis is a well known procedure and can be accomplished under standard conditions well known in the art. Exemplary hydrolysis procedures are described in U.S. Pat. Nos. 3,386,978 and 3,494,908, both of which are incorporated by reference herein.

The rate of diffusion of an active agent from the drug delivery device is broadly determined by measuring the rate of the active agent transferred from one chamber through a sintered glass filter of known pore size and thickness into another chamber and calculating from the obtained data the agent transfer rate. The procedure is well known in the art, and described, for example, in Proc. Roy. Sci. London, Ser. A, 148:1935; J. Pharm. Sci. (1966) 55:1224-1229; and references cited therein. The diffusion coefficient of an active agent can also be experimentally determined by using the same or similar apparatus. Methods for determining the diffusion coefficient are described in Diffusion in Solids, Liquids and Gases, by W. Jost (Rev. Ed., Academic Press Inc. NY; 1960), Chapter XI, pp. 436-488.

The solubility of an active agent in a polymeric material can be determined by various art known techniques. Typical methods used for the measurement of solubility are chemical analysis, measurement of density, refractive index, electrical conductivity, and the like. Details of various methods for determining solubilities are described in U.S. Public Health Service Bulletin No. 67 of the Hygienic Laboratory; Encyclopedia of Science and Technology (McGraw-Hill, Inc.; 1971) 12:542-556; and Encyclopaedic Dictionary of Physics (Pergamon Press, Inc; 1962) 6:545-557. According to Fick's Law, the rate of active agent in solution is directly proportional to the area of the agent, A in cm², as exposed to polymeric material and inversely proportional to the length of the path through which the dissolved active agent must diffuse (see Remington Pharmaceutical Science (Mack Publishing Company, 14th Ed., 1970), pp. 246-269.

For example, the solubility of an active agent in an EVA copolymer may be determined by preparing a saturated solution of the active agent and ascertaining, by analysis, the amount present in a defined area of the copolymer material. For example, the solubility of the active agent in the EVA copolymer is determined by first equilibrating the polymer material with a saturated solution of the active agent at a known temperature, for example 37° C., or with a pure liquid active agent, if the active agent is a liquid at 37° C. Next, the active agent is desorbed from the saturated polymer material with a suitable solvent for the active agent. The resultant solution is then analyzed by standard techniques such as ultraviolet, visible spectrophotometry, refractive index, polarography, electrical conductivity and the like, to calculate the concentration or solubility of the active agent in the material.

The polymeric mixture utilized in the drug delivery device used in the methods of the present invention can be manufactured by standard techniques provided that such manufacture includes process steps such as blending, mixing or the equivalent thereof for structurally defining the system comprising the active agents) to be released and the polymeric matrix. For example, one suitable method for making the inventive devices comprises mixing the polymer and an appropriate solvent, thereby to form a casting solution, mixing a known amount of the agent to be released in the casting solution, charging the solution into a mold and then drying the mold, optionally under vacuum, causing the polymer to precipitate in forming the matrix with the agent to be released therein. In an embodiment, a compounder may be used, such as a recirculating batch mixer (e.g., 13 cc Charge, Serial No. J3836, Randcastle Extrusion Systems, Inc. Cedar Grove, N.J. For example, compounding temperatures of about 160 to about 170° F., e.g. 165° about F (for 10 min) for all runs. In some embodiments, this results in substantially no organic solvents residues and may be a scalable process.

Alternatively, the polymer in the form of a powder can be admixed with the agent to be released in the form of a powder and then molded under adequate temperature and pressure to the desired shape, e.g. a ring, through injection, compression, or extrusion. When two or more agents are to be delivered, the foregoing steps of manufacture can be repeated for each individual agent, thus forming, for example, a separate molded polymeric mixture for each agent. The individual molded polymeric mixtures can be cut into pieces of the required length using conventional cutting techniques, thus producing a plurality of uniform segments. The drug delivery device or system for simultaneous delivery of multiple agents, or for delivery, e.g., of an antiandrogen and one or more contraceptive agents can be then assembled by joining together, directly or indirectly, at least one segment of the molded polymeric mixture for each agent to be delivered. The uniform segments can be assembled to form a ring shape, which has a thickness between about 1 mm and about 5 mm. The drug delivery devices of this invention can be manufactured in a wide range of shapes, sizes and forms for delivering the active agents(s) to different environments of use.

Alternatively, when one, two or more active agents are to be delivered, each active agent:polymer mix can be molded together under adequate temperature and pressure to the desired shape, through injection, compression, or extrusion such that the one or two agent mixtures form one solid unit and do not require a coupling means. In one embodiment, the agent mixtures are injected, preferably sequentially, into a mold comprising a single port. In an alternative embodiment, the active agent mixtures are injected simultaneously or sequentially into a mold having multiple ports. Multiple port moldings are well known and commercially available in the art. Such molding may be modified or customized for a particular application as will be appreciated by those of skill in the art.

In another embodiment, the ends of the segments are joined together to form a drug delivery device using a coupling means. The coupling means can be any method, mechanism, device or material known in the art for bonding materials or structures together. Exemplary coupling means include solvent bonding, adhesive joining, heat fusing, heat bonding, pressure, and the like. When a solvent is used, the ends of the segments are moistened with an organic solvent that causes the surfaces to feel tacky, and when placed in contact the surfaces then bond and adhere in a fluid tight union. The ends of the segments can be adhesively united to form a ring-shaped delivery device by applying an adhesive to at least one end of a segment, and then contacting the adhesive coated end or ends. For the above procedures, the solvents include organic solvents such as methylene chloride, ethylene dichloride, trichlorobenzene, dioxan, isophorone, tetrahydrofuran, aromatic and chlorinated hydrocarbons, mixed solvents such as 50/50 ethylene dichloride/diacetone alcohol; 40/60 alcohol/toluene; 30/70 alcohol/carbon tetrachloride, and the like. Suitable adhesives include natural adhesives and synthetic adhesives, such as animal, nitrocellulosic, polyamide, phenolic, amino, epoxy, isocyanate, acrylic, silicate, organic adhesives of polymers, and the like. Adhesives are well known to the art (see, e.g., The Encyclopedia of Chemistry (Second ed.; G. L. Clark and G. G. Hawley, editors; VanNostrand Reinhold Co., Cincinnati, Ohio; 1966), as well as solvents (see, e.g., Encyclopedia of Chemical Technology (Kirk-Othmer, Sec. Ed., Vol. 16, Interscience, Publishers Inc., New York, 1969)).

The lengths of the segments of the drug delivery device or system can be chosen to give the required performance. Ratios of the lengths of the segments will depend upon the particular therapeutic application, including the desired ratio and dosages of each active agent to be delivered. Ratios of the lengths of the segments are contemplated to be between 30:1 and 1:30, for example between about 15:1 and 1:1. When placebo segments are required to prevent active agent diffusion and interactions, e.g. when two or more active agents are used, the lengths of the placebo segments are long enough to prevent excessive mixing of the active agents. The length of the placebo segment depends on the nature of the polymeric substance and its capacity to prevent permeation of the active agents. In one embodiment, the placebo segment completely or substantially prevents mixing of the active agents, since mixing may disturb the release pattern. However, depending upon which active agent is used, some minor mixing is generally permitted, provided it does affect the release of the active agents in such a manner that plasma levels of the active agents do not substantially exceed the required values.

In an alternate embodiment, polymeric shapes of a drug delivery device are manufactured by preparing unitary segments then joining the ends of the segments to form a ring-shaped drug delivery device for release of one or multiple active agents. Alternatively, the polymeric mixture may be molded into over-sized cylindrical rods, which are then cut into shorter rods having the required dimensions.

The intravaginal drug delivery device used in the methods of the present invention can be manufactured in any size as required. The cross sectional diameter of polymer rods will typically be between about 0.5 mm and 12 mm, between 0.5 mm and 10 mm, between 1 mm and 8 mm, or even between 1 and 6 mm, for example between 1 and 5 mm. In the case of human use, the ring-shaped device has an outer diameter from about 40 mm to about 80 mm; the cross sectional diameter is preferably between about 0.5 mm to 12 mm.

In an exemplary embodiment, provided herein is a vaginal ring having about 15 to about 18 g of EVA (e.g. about 17 g) (for example, about 8.5 grams of EVA having about 28 weight percent vinyl acetate, and a melt index at 190° C./2.16 kg of 28 g/10 mn, and about 8.5 grams of EVA having about 18 weight percent weight percent vinyl acetate, and a melt index at 190° C./2.16 kg of about 150 g/10 mn), and about 1.67% grams of PEG having a wt. average molecular weight of about 4000, about 0.2 grams of Tween 80, and about 0.7 grams of leuprolide acetate.

In some embodiments, disclosed segments or rings may have substantially no vinyl acetate monomers, e.g. such rings may have less than about 1, 0.5, or even less than abut 0.05 weight percent vinyl acetate monomer.

### Methods

In another aspect, the invention relates to a method for vaginally delivering therapeutic agents to a female mammal. The method involves preparing a drug delivery device, as described above. The device is then positioned in the vaginal tract of the female mammal to be treated, where it is maintained for a period of time sufficient to deliver a pharmaceutically effective amount of one or more active agents the female mammal. In one aspect, the pharmaceutically effective amount of one or more active agents is less than the pharmaceutically effective amount when said one or more active agents is administered to a patient orally. In one embodiment, the disclosed method can result in reduced incidence of adverse side-effects in patients as compared to oral administration. In one embodiment, the disclosed method can result in reduced incidence of gastrointestinal side effects in patients as compared to oral administration.

The disclosed method may allow direct administration of the one or more active agents to a target organ without initial metabolism by the liver. For example, an adverse drug interaction may involve alterations of enzymatic activity, e.g. cytochrome P450 enzymatic activity in the liver. For example, if one active agent inhibits a cytochrome P450-mediated metabolism of another active agent, the second active agent may accumulate within the body to toxic levels, possibly causing an overdose. Because the disclosed methods vaginally deliver one or more active agents, the delivery may result in reduced incidence of adverse drug interaction by, e.g., circumventing the initial metabolism of one or more active agents by the liver.

The methods disclosed herein contemplates treating and/or ameliorating obesity, diabetes, multiple sclerosis, endometriosis, polycystic ovarian disease, uterine fibroids, breast cancer, hirsutism, acne, microbial infections (e.g. bacterial vaginosis), coronary heart disease, chronic obstructive pulmonary disease, asthma, chronic kidney disease, or migraine. In some embodiments, the disclosed methods are intended to provide a continuous, simultaneous delivery of physiological combinations of therapeutic agents without the need for injections and/or vaginal gels or creams.

A dose range of a therapeutic agent will depend upon the particular composition used. As will be understood by one of skill in the art, the effective dose ranges will be agent specific and will depend upon patient characteristics, such as species, age and weight. An effective dose range may be determined by routine testing by one of skill in the art, without undue experimentation. For example, an effective dose of one or more contraceptive agents may together provide substantial protection from pregnancy. In another example, an effective dose of one or more cholesterol lowering agents may together provide substantial reduction of blood cholesterol levels.

In one embodiment, the invention relates to a method for treating and/or ameliorating obesity in female mammals (patients) comprising vaginally delivering an effective amount of a therapeutic peptide to a patient in need thereof, e.g. by inserting or positioning into the vaginal tract of a female patient, a drug delivery device as described above that includes a satiety-inducing therapeutic peptide, e.g. a peptide selected from the group consisting of glucagon-like peptide-1 (GLP-1), gastric inhibitory peptide (GIP), ghrelin, oxyntomodulin, peptide YY, pancreatic polypeptide, and amylin. The method of the invention may be initiated at any time following determination of obesity status.

In another embodiment, the invention relates to a method for treating and or ameliorating diabetes in female mammals (patients) in need thereof comprising administering vaginally, a pharmaceutically effective amount of GLP-1 analogue, e.g., exenatide, for example, e.g. by inserting or positioning into the vaginal tract of a female patient, a drug delivery device as described above that includes a therapeutic peptide suitable for treatment of diabetes.

In another embodiment, the invention relates to a method for treating and/or ameliorating multiple sclerosis in female mammals (patients) comprising administering vaginally a pharmaceutically effective amount of glatiramer acetate, for example, e.g. by inserting or positioning into the vaginal tract of a female patient, a drug delivery device as described above that includes glatiramer acetate.

For example, the methods for treating and/or ameliorating multiple sclerosis comprise providing a drug delivery device having at least one segment, wherein at least one of the segment comprises a uniform mixture of peptide-permeable polymeric substance, e.g. ethylene-vinyl acetate co-polymer, and glatiramer acetate. The drug delivery device is then inserted into the vagina of the female mammal and maintained in the vaginal tract for a period of time sufficient to deliver a pharmaceutical effective amount of glatiramer acetate to the female patient. The peptide-permeable polymeric substance may be a thermoplastic polymer, such as an ethylene-vinyl acetate copolymer, and the segment may be shaped as a ring or form part of a ring.

Also contemplated herein are methods for treating acromegaly and diarrhea comprising providing a drug delivery device having at least one segment, wherein at least one of the segment comprises a uniform mixture of peptide-permeable polymeric substance, e.g. ethylene-vinyl acetate co-polymer, and octreotide (or a salt thereof). The drug delivery device is then inserted into the vagina of the female mammal and maintained in the vaginal tract for a period of time sufficient to deliver a pharmaceutical effective amount of octreotide to the female patient.

In one embodiment, the invention relates to a method for treating and/or ameliorating endometriosis or polycystic ovarian disease in female mammals (patients) comprising vaginally delivering an effective amount of a therapeutic peptide (e.g. leuprolide) to a patient in need thereof, e.g. by inserting or positioning into the vaginal tract of a female patient, a drug delivery device as described above.

In another embodiment, the invention relates to a method for treating and or ameliorating uterine fibroids in female mammals (patients) in need thereof comprising administering vaginally, a pharmaceutically effective amount of a therapeutic peptide, e.g. leuprolide, for example, e.g. by inserting or positioning into the vaginal tract of a female patient, a drug delivery device as described above.

In another embodiment, the invention relates to a method for treating breast cancer in female mammals (patients) comprising administering vaginally a pharmaceutically effective amount of e.g. leuprolide acetate, for example, e.g. by inserting or positioning into the vaginal tract of a female patient, a drug delivery device as described above

For example, the methods for treating and/or ameliorating endometriosis and/or polycystic ovarian disease comprise providing a drug delivery device having at least one segment, wherein at least one of the segment comprises a uniform mixture of peptide-permeable polymeric substance, e.g. ethylene-vinyl acetate co-polymer, and leuprolide acetate. The drug delivery device is then inserted into the vagina of the female mammal and maintained in the vaginal tract for a period of time sufficient to deliver a pharmaceutical effective amount of a therapeutic protein, e.g. leuprolide acetate to the female patient. The peptide-permeable polymeric substance may be a thermoplastic polymer, such as an ethylene-vinyl acetate copolymer, and the segment may be shaped as a ring or form part of a ring. In some embodiments, the provided vaginal rings deliver a pharmaceutically sufficient amount transvaginally of e.g. leuprolide acetate to induce substantially complete suppression of the pituitary-gonadal axis. Disclosed rings may deliver leuprolide substantially continuously and may achieve serum levels of about 10 to about 100 ng/ml in a patient.

In one embodiment, the invention relates to a method for treating and/or ameliorating acne in female mammals (patients) comprising vaginally delivering an effective amount of a isotretinoin and at least one contraceptive agent to a patient in need thereof, e.g. by inserting or positioning into the vaginal tract of a female patient, a drug delivery device as described above that includes isotretinoin and at least one contraceptive agent.

In another embodiment, the invention relates to a method for treating and or ameliorating hirsutism in female mammals (patients) in need thereof comprising administering vaginally, a pharmaceutically effective amount of an antiandrogen and a contraceptive agent(s), for example, e.g. by inserting or positioning into the vaginal tract of a female patient, a drug delivery device as described above that includes an antiandrogen suitable for treatment of hirsutism.

In another aspect, the invention relates to a method for vaginally delivering a combination of antiviral agents to a female mammal. The method involves preparing a drug delivery device, as described previously. The device is then positioned in the vaginal tract of the female mammal to be treated, where it is maintained for a period of time sufficient to deliver a pharmaceutically effective amount of the antiviral agents to the female mammal. The disclosed methods of preventing e.g. HIV are, in some embodiments, intended to provide a continuous, simultaneous delivery of a combination of antiviral agents and may provide prophylactic protection against viral infection.

For example, provided herein is a method for treating and/or ameliorating bacterial vaginosis in female mammals (patients) in need thereof comprising administering vaginally, a pharmaceutically effective amount of an antibacterial agent by inserting or positioning into the vaginal tract of a female patient, a drug delivery device as described above that includes antibacterial agents suitable for treatment of bacterial vaginosis.

In one embodiment, the invention relates to a method for HIV prophylaxis in female mammals (patients) in need thereof comprising providing a drug delivery device having at least one segment, wherein at least one segment comprises a uniform mixture of drug-permeable polymeric substance, e.g. ethylene-vinyl acetate copolymer, and an antiviral agent or combination of antiviral agents e.g., tenofovir and UC781. The drug delivery device is then inserted into the vagina of the female mammal and maintained in the vaginal tract for a period of time sufficient to deliver a pharmaceutically effective amount of the antiviral agents to the female patient. The drug-permeable polymeric substance may be a thermoplastic polymer, such as an ethylene-vinyl acetate copolymer, and the segment may be shaped as a ring or form part of a ring.

In one embodiment, the disclosure relates to a method for treating and/or ameliorating coronary heart disease in female mammals (patients) in need thereof comprising administering vaginally, a pharmaceutically effective amount of a cholesterol lowering agent, beta-blocker, nitroglycerin, calcium channel blocker, aspirin, or a combination thereof by inserting or positioning into the vaginal tract of a female patient, a drug delivery device as described above that includes active agents suitable for the treatment of coronary heart disease.

In another embodiment, the disclosure relates to a method for treating and/or ameliorating chronic obstructive pulmonary disease or asthma in female mammals (patients) in need thereof comprising administering vaginally, a pharmaceutically effective amount of a bronchodilator, antibiotic, and a combination thereof by inserting or positioning into the vaginal tract of a female patient, a drug delivery device as described above that includes active agents suitable for the treatment of chronic obstructive pulmonary disease or asthma.

In yet another embodiment, the disclosure relates to a method for treating and/or ameliorating chronic kidney disease in female mammals (patients) in need thereof comprising administering vaginally, a pharmaceutically effective amount of ACE inhibitor, angiotensin II receptor antagonist, or a combination thereof by inserting or positioning into the vaginal tract of a female patient, a drug delivery device as described above that includes active agents suitable for the treatment of chronic kidney disease.

In a further embodiment, the disclosure relates to a method for treating and/or ameliorating migraine in female mammals (patients) in need thereof comprising administering vaginally, a pharmaceutically effective amount of an anti-nausea drug, analgesic, or a combination thereof by inserting or positioning into the vaginal tract of a female patient, a drug delivery device as described above that includes active agents suitable for the treatment of migraine.

Also contemplated herein are methods for treating and/or ameliorating two or more chronic disorders simultaneously, e.g. a method of treating COPD and heart disease, comprising administering vaginally a pharmaceutically effective amount of e.g. a COPD treating agent and a heart disease treatment agent, by inserting or positioning into the vaginal tract of a female patient a drug delivery device as described above that includes one or more active agents suitable for the treatment of one disease, and one or more active agents suitable for the treatment of a different disease.

Contemplated herein, in some embodiments, is administration of therapeutic agents in a dose regimen that may include inserting a disclosed therapeutic ring having a first amount therapeutic agent and leaving that in place for a period of time, removing that ring, and optionally inserting a disclosed therapeutic ring having a second amount of the same therapeutic agent, or a different therapeutic agent. In an different embodiment, substantially simultaneous administration of two therapeutic agents, e.g. a therapeutic peptide and another therapeutic agent can be accomplished, for example, by administering to the subject a ring having a fixed ratio of each therapeutic agent and/or peptide. In another embodiment, sequential or substantially simultaneous administration of another therapeutic agent (upon e.g. insertion of a vaginal ring having a therapeutic agent) can be effected by any other appropriate route including, but not limited to, oral routes, intravenous routes, intramuscular routes, and direct absorption through mucous membrane tissues.

The following examples are merely illustrative of the present invention and they should not be considered as limiting the scope of the invention in any way, as these examples and other equivalents thereof will become apparent to those versed in the art in the light of the present disclosure, drawings and the accompanying claims

### EXAMPLES

### Example 1

An intravaginal drug delivery device illustrated in FIG. 1B is prepared using poly(ethyl-co-vinyl acetate) (EVA) manufactured by Aldrich Chemical Co. (Cat. No. 34,050-2; Lot No. 07322DR). Isotretinoin is dissolved or distributed in approximately 5 mL of dichloromethane (Fluka Chem. Co.; Cat. No. 66740; Lot No. 404915/1 62800) in a scintillation vials. Next, polymeric mixtures are prepared by adding 1400 mg of EVA to the solution and mixing the EVA/drug compositions using a rotary shaker. The resulting mixtures are then solvent cast in dry ice using ethanol as the solvent (Pharmco; Cat. No. 111 USP 200 CSGL; Lot No. M8241). The solvent is allowed to evaporate overnight, and the dry EVA/drug mixtures are then ground into powders.

The EVA/drug powders are placed in an injection molding unit (DSM, Geleen, Holland). The injector is heated to approximately 80° C. The molten EVA/drug compositions are extruded into stainless steel mold at 10 C, creating a 1800 mg ring with an outer diameter of 50 mm and a cross section of 4 mm.

### Example 2

An intravaginal drug delivery device is prepared using poly(ethyl-co-vinyl acetate) (EVA) manufactured by Aldrich Chemical Co. (Cat. No. 34,050-2; Lot No. 07322DR). Metronidazole is dissolved or distributed in approximately 5 mL of dichloromethane (Fluka Chem. Co.; Cat. No. 66740; Lot No. 404915/1 62800) in a scintillation vials. Next, polymeric mixtures are prepared by adding 1400 mg of EVA to the solution and mixing the EVA/drug compositions using a rotary shaker. The resulting mixtures are then solvent cast in dry ice using ethanol as the solvent (Pharmco; Cat. No. 111 USP 200 CSGL; Lot No. M8241). The solvent is allowed to evaporate overnight, and the dry EVA/drug mixtures are then ground into powders.

The EVA/drug powders are placed in an injection molding unit (DSM, Geleen, Holland). The injector is heated to approximately 80° C. The molten EVA/drug compositions are extruded into stainless steel mold at 10 C, creating a 1800 mg ring with an outer diameter of 50 mm and a cross section of 4 mm.

### Example 3

An intravaginal drug delivery device is prepared using poly(ethyl-co-vinyl acetate) (EVA) manufactured by Aldrich Chemical Co. (Cat. No. 34,050-2; Lot No. 07322DR). Atorvastatin is dissolved or distributed in approximately 5 mL of dichloromethane (Fluka Chem. Co.; Cat. No. 66740; Lot No. 404915/1 62800) in a scintillation vials. Next, polymeric mixtures are prepared by adding 1400 mg of EVA to the solution and mixing the EVA/drug compositions using a rotary shaker. The resulting mixtures are then solvent cast in dry ice using ethanol as the solvent (Pharmco; Cat. No. 111 USP 200 CSGL; Lot No. M8241). The solvent is allowed to evaporate overnight, and the dry EVA/drug mixtures are then ground into powders.

The EVA/drug powders are placed in an injection molding unit (DSM, Geleen, Holland). The injector is heated to approximately 80° C. The molten EVA/drug compositions are extruded into stainless steel mold at 10 C, creating a 1800 mg ring with an outer diameter of 50 mm and a cross section of 4 mm.

### Example 4

150.0 g of a EVA polymer (with 40% vinyl acetate content) (Evatane 40-55®) was weighed into a weighing pan. Next, 1 L of de-ionized water was placed in a 2-L beaker and was stirred water by Arrow Engineering Mixer using setting of 2.5. The polymer was charged into batch while mixing. The copolymeric beads were stirred in water for 20 minutes. After stirring, the material was filtered and collected. Evatane® beads were washed 9 times by repeating the cycle of washing and filtration. Following wash-filter cycle, collected wash-filtered beads were further drained by passing the copolymeric material through a 1700-microns sieve. Finally, polymeric material, along with the sieve, was placed in a vacuum oven for 48 hours.

The intravaginal drug delivery device illustrated in FIG. 1A is prepared with the poly(ethyl-co-vinyl acetate) (EVA) prepared as above. 18 mg, 36 mg, or 54 mg leuprolide acetate is dissolved or distributed in approximately 10 mL of ethanol in scintillation vials. Next, polymeric mixtures are prepared by adding 1400 mg of EVA to the solution and mixing the EVA/drug compositions using a rotary shaker. The resulting mixtures are then solvent cast in dry ice using ethanol as the solvent (Pharmco; Cat. No. 111 USP 200 CSGL; Lot No. M8241). The solvent is allowed to evaporate overnight, and the dry EVA/drug mixtures are then ground into powders.

The EVA/drug powders are placed in an injection molding unit (DSM, Geleen, Holland). The injector is heated to approximately 80° C. The molten EVA/drug compositions are extruded into stainless steel mold at 10 C, creating a ring with an outer diameter of 50 mm and a cross section of 4 mm.

### Example 5

A three day study is conducted to investigate systemic absorption of leuprolide acetate delivered via a vaginal ring. A vaginal ring prepared as in Example 4 with 18 mg leuprolide acetate (low dose) is inserted in vaginally in female patients at 8 am of day 1 and removed at 4 pm of day 3. Before the vaginal ring is inserted, blood is drawn and analyzed to determine pretreatment levels of gonadotropins and sex steroid. After the vaginal ring is inserted, blood is drawn at 8 am, noon and 4 pm on each of the three days. Women studied were in early follicular phase (1^{st} week of menses) to keep endogenous levels of E₂ and progesterone levels at nadir.

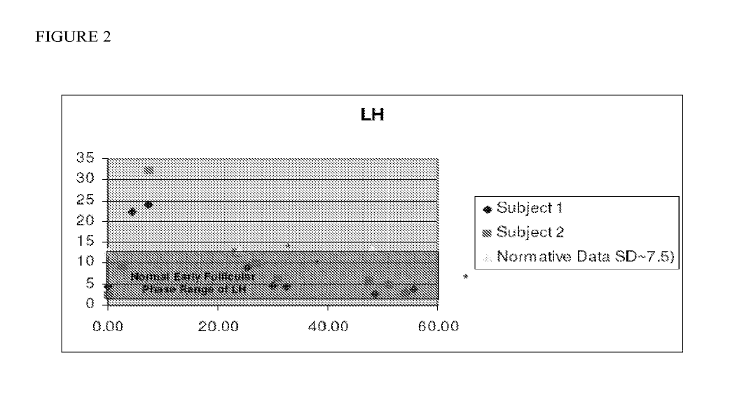

FIGS. 2, 3, 4, and 5 depict results from 2 subjects. Subject 1 was studied towards end of menses and subject 2 at beginning of menses. Some specimens of subject 2 were hemolyzed (marked by *) which did not seem to have a significant effect. The normative data shown is based on approximately 90 women in early menses.

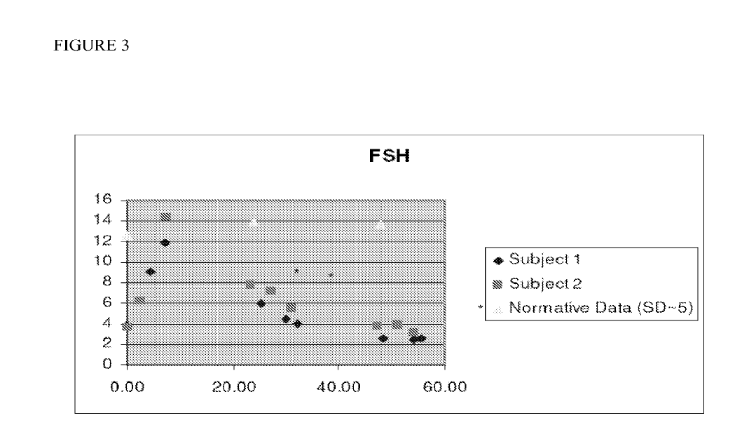

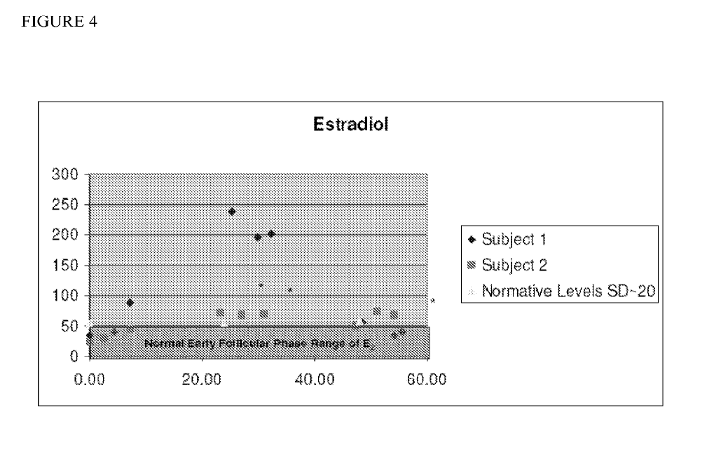

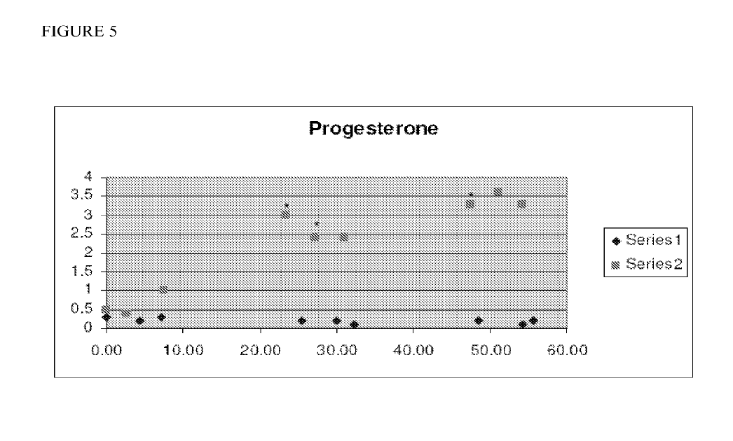

FIG. 2, FIG. 3, FIG. 4, and FIG. 5 depict, respectively, LH response (miU/ml), FSH response (miU/ml), estradiol response (pg/ml), and progesterone response (ng/ml) in the two subjects using transvaginal delivery of leuprolide with a ring of Example 4. The x axis of each Figure depicts hours after ring insertion.

### Example 6

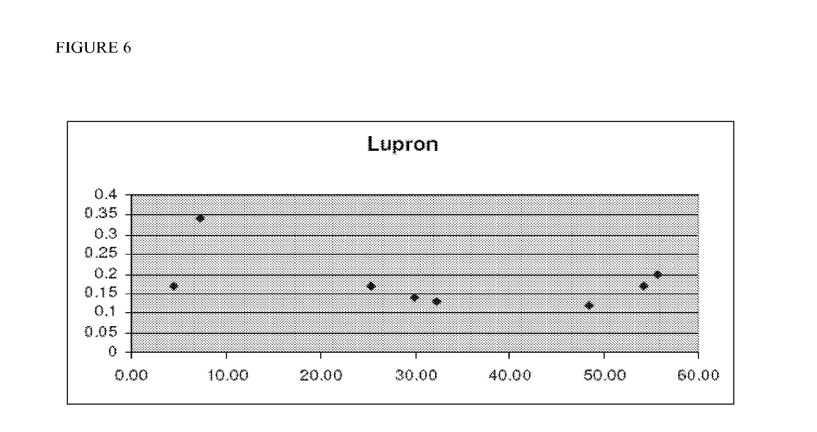

FIG. 6 depicts results of mass spectra analysis on the leuprolide levels of subject 1 during the 3 day trial described in Example 5.

### Example 7

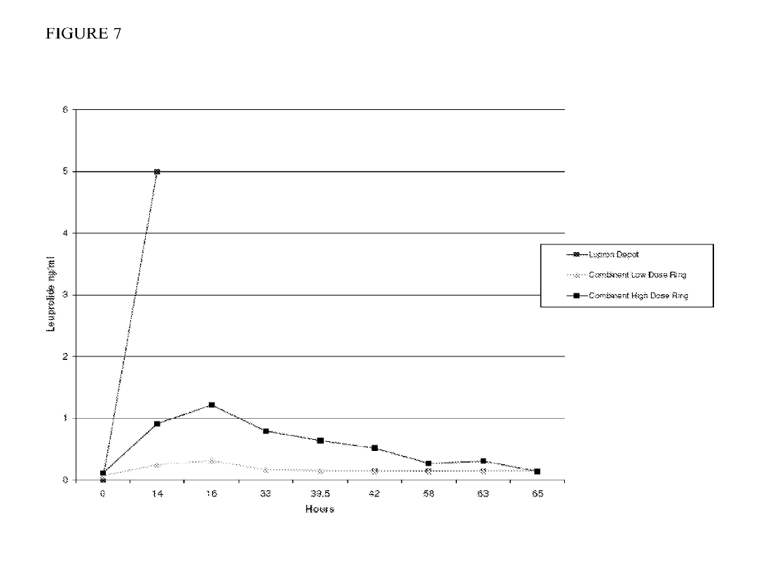

A three day study with six patients is conducted to investigate systemic absorption. A vaginal ring prepared as in Example 4 with 18 mg leuprolide acetate (low dose) is inserted into three female patients at 8 am of day 1 and removed at 4 pm of day 3, and a vaginal ring with 36 mg leuprolide (high dose) is inserted into three other female patients at 8 am of day 1 and removed at 4 pm of day 3. Blood is drawn at 8 am, noon and 4 pm on each of the three days. FIG. 7 depicts a comparison of average leuprolide levels in patients administered the high dose and the low dose of leuprolide via the ring as compared to a patient administered Lupron® Depot (22.5 mg), and depicts a comparison of average serum level of leuprolide of patients administered the high dose vs. the low dose of leuprolide. Although the 36 mg ring (high dose) has double the dose of the 18 mg ring (low dose), the average peak serum level of the high dose patients is more than two times the peak serum level of the “low dose” patients.

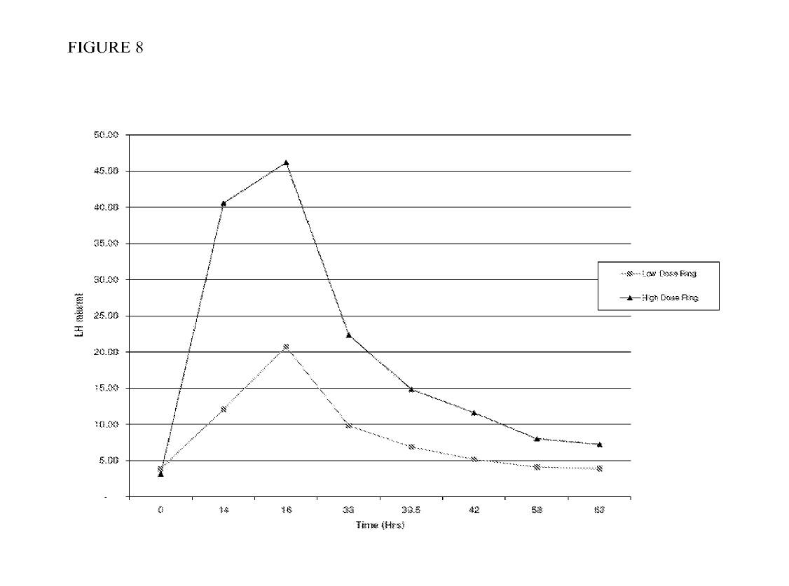

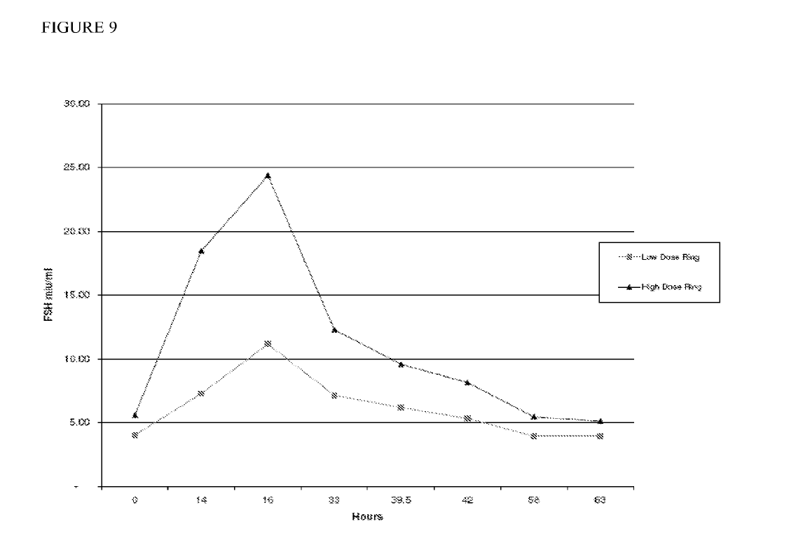

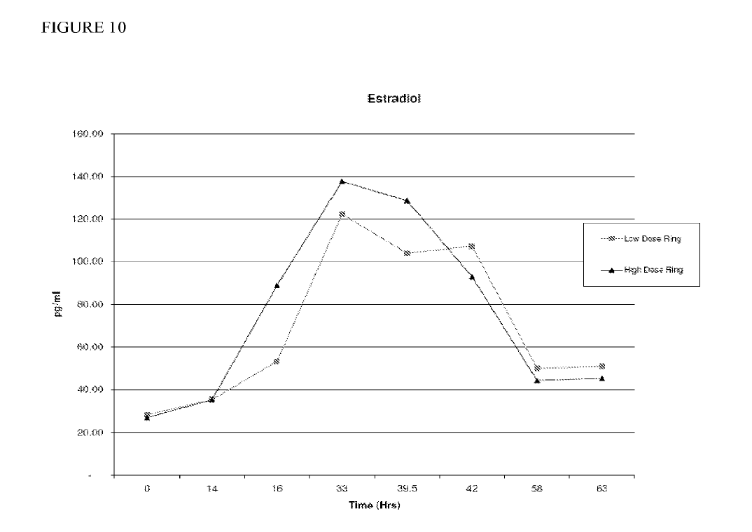

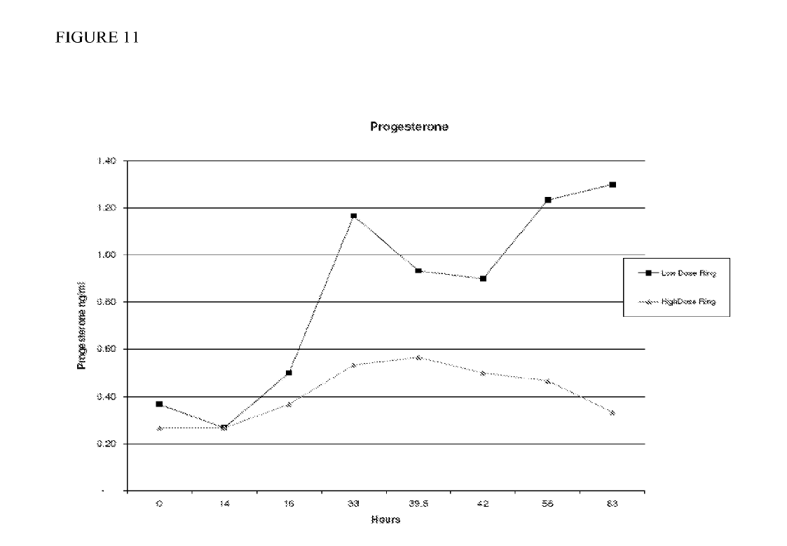

FIG. 8, FIG. 9, FIG. 10, and FIG. 11 depict, respectively, the average LH response (miU/ml), FSH response (miU/ml), estradiol response (pg/ml), and progesterone response (ng/ml) of the subjects using transvaginal high or low dose delivery of leuprolide with a ring. The x axis represents of each of these Figures depicts hours after ring insertion.

### Example 8

Tampons which undergo both radial and longitudinal expansion are generally manufactured for use with various types of applicator devices. The longitudinal expansion is generally more than 10% of its unexpanded length. This type of tampon comprises an inner core of absorbent material, such as cellulose fibers and/or cotton fibers, enveloped by an outer layer of liquid-permeable material such as a non-woven polymer, as for example polypropylene, polyethylene, polyester, cellulose, cellulose derivatives, or any combination of the above.

In this example, a delivery system according to the invention in the form of three segmented cylindrical EVA polymer are positioned between the outer layer and the inner core, in parallel to the longitudinal axis of the body. The length of the segmented EVA cylinders may be equal to the length of the tampon. Each EVA cylinder is composed of 3 segments. The first segment contains 10 mg of Leuprolide (luteinizing hormone-releasing hormone agonist) the second segment does not contain any drug and the third segment contains 1.5 g of progesterone.

Upon insertion of the tampon the drugs are being released from the segmented EVA cylinders. Each drug is released from its own segment with its own release kinetics to the surrounding environment.

### Example 9

Three segmented EVA cylinders are positioned at spaced intervals between the outer layer and the inner core of the tampon, perpendicularly to the longitudinal axis of the tampon body. The length of each of the strips is equal to the length of the tampon.

### Example 10

### Preparation of a Drug Delivery Tampon for the Controlled Release of Rogesterone, Estradiol and Gonadotropin Releasing Hormone (GnRH)

The intravaginal drug delivery device illustrated in FIG. 1 is prepared using poly(ethyl-co-vinyl acetate) (EVA) manufactured by Aldrich Chemical Co. (Cat. No. 34,050-2; Lot No. 07322DR). 630 (milligrams) mg of progesterone (Sigma Corp.; Cat. No. P-3972) or 2.8 mg of estradiol (Sigma Corp.; Cat. no. E-1072) are each dissolved separately in approximately 5 mL of dichloromethane (Fluka Chem. Co.; Cat. No. 66740; Lot No. 404915/1 62800) in separate scintillation vials. Next, polymeric mixtures are prepared by adding 1400 mg of EVA to the progesterone solution and 1800 mg of EVA to the estradiol solution, and mixing the EVA/drug compositions using a rotary shaker. The resulting mixtures are then solvent cast in dry ice using ethanol as the solvent (Pharmco; Cat. No. 111 USP 200 CSGL; Lot No. M8241). The solvent is allowed to evaporate overnight, and the dry EVA/drug mixtures is then ground into powders. The EVA/drug powders are placed in an injection molding unit (DSM, Geleen, Holland). The injector is heated to approximately 80° C. The molten EVA/drug compositions are extruded into stainless steal mold (the mold is at 10° C.), creating a 1800 mg cylinder with a cross section of 4 mm and 20 cm in length.

Similarly, a polymeric segment comprising GnRH agonist (D-Trp6-Pro9-Net-GnRH) is prepared by dissolving 10 mg of GnRH agonist and 450 mg of methyl cellulose in approximately 5 mL methylene chloride. 1800 mg EVA is added. The EVA/drug mixture is dried and the resulting powder is placed in an injection molding unit (DSM, Geleen, Holland). The injector is heated to approximately 80° C. The molten EVA/GnRH/cellulose compositions are extruded into stainless steel mold (the mold is at 10° C.), creating a 1800 mg EVA cylinder with a cross section of 4 mm and 20 cm in length.

The EVA segments containing estradiol, progesterone, and GnRH are each aseptically cut into unitary cylindrical segments of the appropriate lengths, i.e., lengths that contain sufficient quantities of drug to provide therapeutically effective amounts of each drug. The pieces of the EVA loaded drug are put together using radio frequency welding.

The segmented EVA rod described herein is designed to deliver 21-day dosages of each drug, i.e., 100 micrograms (μg) per day of estradiol, 6 milligrams (mg) per day of progesterone, and 240 μg per day of GnRH.

### Example 11

An ethylene vinyl acetate based ring having PEG4000 is prepared as follows, using the following materials:

| Material | Source | Amount (grams) |
|---|---|---|
| EVA | Arkema | 18.04 |
| PEG4000 | Dow Chemicals | 1.76 |
| Tween 80, Pharma | Corda | 0.20 |

A 50:50 mixture of Evatane® 18-150 EVA (a random copolymer of ethylene and vinyl acetate, with about 18% by weight vinyl acetate content, and a melt index of about 150 g/10 min) with Evatane® 28-30 or 28-25 EVA (a random copolymer of ethylene and vinyl acetate with about 28% by weight vinyl acetate content, and a melt index of about 25 g/10 min) is used.

The EVA is washed by adding 150 g of EVA beads to a 1 lit of USP or Milli-Qwater at room temperature, and stirred vigorously for 20 minutes. The EVA beads are then filtered and this washing process is repeated 10 times. The polymer is then dried in a vacuum oven at room temperature for 48 hours. A headspace GC-FID (Primera/Bionex PASC-TMS-0009) was used to detect any free vinyl acetate. No detectable residues of free vinyl acetate were present.

The materials were then compounded by first mixing EVA, PEG and Tween manually. The homogenous mixture is added to the compounder for 10 min at 65 RPM and at 165° F. (74° C.). After cooling the solid mixture is chopped to small pieces and added to an injection molding barrel. The final ring is depicted in FIG. 1C.

Quality control acceptance criteria are applied, i.e.: Appearance—smooth round ring; Color—clear to white; Measurements: OD=54±0.37 mm; ID=46±0.31 mm; d=4±0.15 mm

The flexibility of the EVA Ring was determined and compared with the flexibility of Nuvaring. The flexibility was determined by means of a press-pull apparatus (such as LR 5K, Lloyd Instruments or TA-XTPlus Texture Analyser). The entire ring-in a relaxed state was fixed in two V-shaped holders. The distance between the corners of the V-shaped profiles is 54 mm. Subsequently the holders were pressed towards each other with a predetermined-speed of 50 mm/min until the distance between the corners of the V-shaped profiles was 21 mm. The force in Newton that was applied to—the ring-shaped drug delivery system to bring about a certain deformation of the ring was measured at predetermined spots: 10 mm (i.e., at a distance of 44 mm), 20 mm (i.e., at a distance of 34 mm), 30 mm (i.e., at a distance of 24 mm) and 33 mm (i.e., at a distance of 21 mm).

The flexibility of the prepared ring is as follows:

| Deformation | Force | STDEV (±) |
|---|---|---|
| (mm) | (gram) | (gram) |
| 10 | 165 | 25 |
| 20 | 275 | 40 |
| 30 | 370 | 55 |
| 33 | 390 | 60 |

### Example 12

An leuprolide ring is prepared following Example 11, using the following materials:

| Material | Source | Amount (grams) |
|---|---|---|
| EVA | Arkema | 17.15 |
| PEG 4000 | Dow Chemicals | 1.67 |
| Tween 80, Pharma | Corda | 0.22 |
| Leuprolide | PolyPeptide | 0.68 |

A 50:50 mixture of Evatane® 18-150 EVA (a random copolymer of ethylene and vinyl acetate, with about 18% by weight vinyl acetate content) with Evatane® 28-30 or 28-25 EVA (a random copolymer of ethylene and vinyl acetate with about 28% by weight vinyl acetate content) is used.

Leuprolide (Glp-His-Trp-Ser-Tyr-Gly-Leu-Arg-Pro-Gly-NH2) was purchased from PolyPeptide Laboratories as GMP grade, Acetate Salt

The EVA is washed by adding 150 g of EVA beads to a 1 lit of USP or Milli-Qwater at room temperature, and stirred vigorously for 20 minutes. The EVA beads are then filtered and this washing process is repeated 10 times. The polymer is then dried in a vacuum oven at room temperature for 48 hours. A headspace GC-FID (Primera/Bionex PASC-TMS-0009) was used to detect any free vinyl acetate. No detectable residues of free vinyl acetate were present.

The materials were then compounded by first mixing the EVA, PEG, Tween and leuprolide manually in a 50 cc beaker. The material amounts are shown below:

| | Amount | Amount |
|---|---|---|
| Material | (grams) | (%) |
| EVA 18-150 | 8.58 | 43.49% |
| EVA 28-30 | 8.58 | 43.49% |
| PEG 4000 | 1.67 | 8.46% |
| Tween 80 | 0.22 | 1.12% |
| Leuprolide | 0.68 | 3.45% |

The homogenous mixture is added to the compounder for 10 min at 65 RPM and at 165° F. (74° C.). After cooling the solid mixture is chopped to small pieces and added to the injection molding barrel. Quality control acceptance criteria are applied, i.e.: Appearance—smooth round ring; Color—clear to white; Measurements: OD=54±0.37 mm; ID=46±0.31 mm; d=4±0.15 mm.

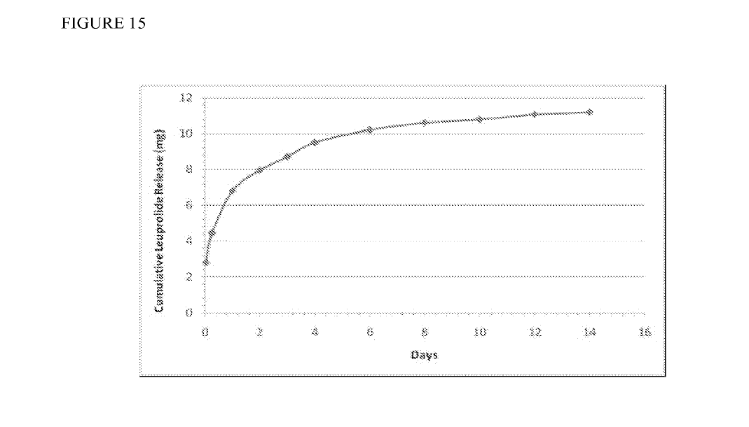

The in-vitro release kinetics are shown in FIG. 15.

The flexibility of the EVA Ring was determined and compared with the flexibility of Nuvaring. The flexibility was determined by means of a press-pull apparatus (such as LR 5K, Lloyd Instruments or TA-XTPlus Texture Analyser). The entire ring-in a relaxed state was fixed in two V-shaped holders. The distance between the corners of the V-shaped profiles is 54 mm. Subsequently the holders were pressed towards each other with a predetermined-speed of 50 mm/min until the distance between the corners of the V-shaped profiles was 21 mm. The force in Newton that was applied to—the ring-shaped drug delivery system to bring about a certain deformation of the ring was measured at predetermined spots: 10 mm (i.e., at a distance of 44 mm), 20 mm (i.e., at a distance of 34 mm), 30 mm (i.e., at a distance of 24 mm) and 33 mm (i.e., at a distance of 21 mm).

The flexibility of the prepared ring is as follows:

| Deformation | Force | STDEV (±) |
|---|---|---|
| (mm) | (gram) | (gram) |
| 10 | 165 | 25 |
| 20 | 275 | 40 |
| 30 | 370 | 55 |
| 33 | 390 | 60 |

### Example 13

The stability of rings that include leuprolide made following Example 4 was investigated. Leuprolide/EVA Rings, 18 mg, were manufactured following Example 4. The rings were stored in stability chambers, under three conditions: 25° C./60% RH; 30° C./60% RH; and 40° C./75% RH, in accordance with ICH guild lines, and samples were pulled and tested at Month 1 and 3 (40° C./75% RH), and Month 6, 9 and 12 (30° C./60% RH). The tests conducted are drug assay in the ring (mg of leuprolide/ring), and related substances of leuprolide (impurities), using validated methods and qualified USP method.

The Assay and impurities test results of Month 0, Month 1, Month 3, Month 6, Month 9 and Month 12 are presented below:

TABLE
Stability study data for Leuprolide/EVA Rings, 18 mg,

| | Potency | Potency | | | |
|---|---|---|---|---|---|
| | % | % | Assay, | Impurities | Impurities |
| | Spec. | Found | mg/ring | Spec. | Found |
| Test Method | TMS- | TMS- | TMS-0008 | TMS-0005 | TMS-0005 |
| | 0008 | 0008 | | | |
| Month 0 | 85-115 | 85.7 | 15.4 | NMT 5%* | ND** |
| Mo. 1, | 85-115 | 90.3 | 16.2 | NMT 5%* | ND |
| 40° C./75% RH | | | | | |
| Mo. 3, | 85-115 | 92.2 | 16.6 | NMT 5%* | ND |
| 40° C./75% RH | | | | | |
| Mo. 6, | 85-115 | 100 | 18.7 | NMT 5%* | ND |
| 30° C./60% RH | | | | | |
| Mo. 9, | 85-115 | 92.5 | 16.7 | NMT 5%* | ND |
| 30° C./60% RH | | | | | |
| Mo. 12, | 85-115 | 94.2 | 17.0 | NMT 5%* | ND |
| 30° C./60% RH | | | | | |

*Total: NMT 5.0%, Individual: NMT 2.0%.
**Non-detectable

Leuprolide/EVA rings, 18 mg, are stable at 40° C./75% RH for at least 3 months. Leuprolide/EVA rings, 18 mg, are stable at 30° C./60% RH for at least 12 months.

### REFERENCES

All publications and patents mentioned herein, including those items listed below, are hereby incorporated by reference in their entirety as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

What is claimed is:

1. A therapeutic device for delivery of a therapeutic peptide via the vagina to treat a disorder affecting a female patient, comprising at least one segment that comprises a peptide permeable thermoplastic polymer and the therapeutic peptide or pharmaceutically acceptable salts thereof, and optionally a pharmaceutically acceptable excipient.

2. The therapeutic device of claim 1, wherein upon insertion of the device in the vagina of said patient results in systemic absorption of said therapeutic peptide.

3. The therapeutic device of claim 1, wherein the therapeutic peptide is a peptide about 4 to about 40 amino acids in length.

4. The therapeutic device of claim 1, wherein the therapeutic peptide is leuprolide or a pharmaceutically acceptable salt thereof.

5. The therapeutic device of claim 1, wherein the therapeutic peptide is chosen from exanatide, liraglutide, oxyntomodulin, ghrelin, peptide YY, pramlintide, and pancreatic polypeptide and combinations thereof.

6. The therapeutic device of claim 1, wherein the therapeutic peptide is chosen from amylin and leptin, and combinations thereof.

7. The therapeutic device of claim 1, wherein the segment is a unitary segment having a substantially uniform composition comprising the therapeutic peptide and the thermoplastic polymer.

8. The therapeutic device of claim 1, wherein about 16 hours or more after insertion of said device, the serum level of said therapeutic peptide is at a pharmaceutically effective amount for the treatment of said disorder.

9. The therapeutic device of claim 8, wherein about 1 day or more after insertion of said device, the serum level of said therapeutic peptide in said patient is at a pharmaceutically effective amount for the treatment of said disorder.

10. The therapeutic device of claim 1, wherein peak serum concentration of the therapeutic peptide after insertion in said patient is obtained at about 12 to about 22 hours after insertion of the device.

11. The therapeutic device of claim 1, wherein peak serum concentration of the therapeutic peptide in said patient is obtained at about 16 hours after insertion of the device.

12. The therapeutic device of claim 4, wherein the peak serum concentration of leuprolide in said patient is less after insertion of said device than that of a patient administered a leuprolide depot composition by injection.

13. The therapeutic device of claim 4, wherein the peak serum concentration of leuprolide in said patient occurs more slowly after insertion of said device as compared to that of a patient administered a leuprolide depot injection by injection.

14. The therapeutic device of claim 12, wherein upon insertion of said device, the serum level of leuprolide in said patient is about 0.05 ng/mL to about 1.5 ng/mL after about 1 day.

15. The therapeutic device of claim 12, wherein upon insertion of said device, the serum level of leuprolide in said patient is about 1.0 ng/mL after about 1 day.

16. The therapeutic device of claim 7, wherein the composition comprises about 10 to about 100 mg of the therapeutic peptide.

17. The therapeutic device of claim 7, wherein the composition comprises about 10 to about 60 mg of leuprolide acetate.

18. The therapeutic device of claim 17, wherein the composition comprises about 36 mg leuprolide acetate.

19. The therapeutic device of claim 17, wherein the composition comprises about 54 mg leuprolide acetate.

20. The therapeutic device of claim 18, wherein the therapeutic device releases about 10 μg/day of leuprolide upon insertion into the vagina of a patient.
